(19) United States
(12) Patent Application Publication
(10) Pub. No.: US 2026/0086056 A1
(43) Pub. Date: Mar. 26, 2026

(54) Tire Defect Detection System that Images Localized Cooling at a Defect

(71) Applicant: Paul E. Hawkinson Company, St. Michael, MN (US)

(72) Inventors: Gary W. Box, Golden Valley, MN (US); Brian B. Brasch, Fargo, ND (US); Anthony S. Rike, Ramsey, MN (US); Jason J. Lawinger, Albertville, MN (US)

(21) Appl. No.: 18/895,560

(22) Filed: Sep. 25, 2024

(51) Int. Cl. G01N 25/72; G01J 5/00; G01N 33/00

(52) U.S. Cl. CPC G01N 25/72; G01J 2005/0077; G01N 33/0085

(57) ABSTRACT

A tire defect detection system for detecting defects in a tire. The system includes at least one infrared camera, a pneumatic source and a computing device. The pneumatic source inflates the tire to a predetermined pressure. After inflation, the infrared camera captures a reference frame of a section of the tire. A period of time after capturing the reference frame, the infrared camera captures a subsequent frame. The subsequent frame is compared to the reference frame to detect a portion of the section of the tire that has a lower temperature. The lower temperature is caused by an escape of air from the tire through a defect. The escaping air cools the area of the tire around the defect, so a decrease in temperature indicates the defect.

### CROSS REFERENCE TO RELATED APPLICATIONS

Not applicable.

### STATEMENT REGARDING FEDERALLY SPONSORED RESEARCH OR DEVELOPMENT

Not applicable to this application.

### BACKGROUND

The described example embodiments in general relate to systems for detecting defects (e.g., flaws) in tires.

Present tire defect detection systems and methods require removing the tire from the vehicle, immersing the tire in a tank (e.g., dunk tank) of liquid (e.g., water), and manually inspecting the tire for bubbles that indicate air leaking from a defect in the tire. A variation of the present systems and methods requires removing the tire from the vehicle, wetting or immersing one or more portions of the tire, and manually inspecting for defects. Present systems and methods are disadvantageous because the water that is used to wet the tire is considered a hazardous waste material that must be disposed of accordingly. Further, the present systems and methods are manually intensive, time consuming, and require trained technicians. Additionally, no record is created regarding the examination of the tire and/or the results of the examination.

Tire manufacturers, installers, retailers and/or consumers may benefit from a tire defect detection system and methods that use a dry process that may be fully automated or performed by a person who is not a trained technician or while the tire is still mounted to the vehicle.

### SUMMARY

Some of the various embodiments of the present disclosure relate to a using thermal (e.g., infrared) images to detect defects in a tire. The infrared images may be captured while the tire is mounted to the vehicle or they may be captured while the tire is removed from the vehicle. Tire defect detection may also be incorporated into a tire inflation cage, wheel balancer, tire spreader, and/or tire changing machine. A tire is deflated by either removing the valve core or automatically via the machine. The tire is then inflated using either hot or cold air. A frame (e.g., image) is captured while after inflation of the tire. Defects generally permit air to escape from the tire.

In some embodiments, the images can be analyzed via blob detection. Blob detection is an image processing technique used to identify regions in an image that differ in properties, such as brightness or color, from their surroundings. In the context of tire defect identification, high-resolution thermal (infrared) or visual images of the tire are captured, highlighting potential defects. These images can be obtained while the tire is mounted on the vehicle or after removal. Software analyzes these images by detecting blobs, or areas that deviate from the norm, indicating possible defects like air pockets, separations, or cracks. The detected blobs are then further examined to assess the nature and severity of the defects, enabling precise identification and localization within the tire.

In some instances, acoustic signals can be used in lieu of, or in combination with, thermal imaging to detect defects in a tire. For example, a tire could be mounted onto a specialized machine, which can be a tire inflation cage, wheel balancer, tire spreader, or tire changing machine. The tire is deflated by either removing the valve core or automatically via the machine. Ultrasonic transducers are placed around the tire to emit and receive ultrasonic waves. The tire is then inflated using either hot or cold air. As the ultrasonic waves travel through the tire, defects such as air pockets, cracks, or separations affect the wave propagation. The received ultrasonic signals are analyzed for anomalies indicating defects. Software processes these signals using techniques like time-of-flight analysis or frequency response analysis to detect and locate defects within the tire. Other examples include acoustic emission testing (AET), sonic resonance testing, impact-echo testing, and the like, all of which are disclosed in greater detail infra.

The tire defect detection system may detect defects in the sidewalls and or the tread of the tire. Generally, the tire defect detection system will divide the tire into sections for analysis. A one or more frames are captured for each section and analyzed to determine whether the section has a defect. The captured frames and the results of analysis may be stored to create a record of the inspection. The system for inspecting the tire for defects may perform all of the steps for inspecting the tire except for positioning the tire on the system and removing the tire from the system. In an embodiment of the system in which the tire is inspected while still connected to the vehicle, a user merely has to drive the tire system support and place the vehicle in neutral. So, the person operates the system to inspect a tire requires little or no training and does not need to maintain a record of the test because the system creates and stores the record.

Some of the various embodiments of the present disclosure include a computing device, a pneumatic source, a tire positioner and an infrared camera. Other example embodiments of the present disclosure include a computing device, a pneumatic source, a tire positioner, a heat source, a support, a temperature sensor, a display, a server, a database, and one or more infrared cameras. Various embodiments of the present disclosure are fully automated such that the operator need merely to place the tire on the system and remove the tire from the system after testing. Other systems require the user to control the pneumatic source, manually position the tire and manually position the infrared camera. Another example embodiment does not require the tire to be removed from the vehicle for testing. In another example embodiment, the entire tire (e.g., all sections) are inspected at the same time. None of the systems require that the operator be trained to detect defects.

In some example embodiments of the present disclosure, the tire defect inspection system captures the data, analyzes the data, presents images of the data on a display, presents the results of the inspection on the display, creates a record of the inspection and stores the record of the inspection. In another example embodiment, the tire defect inspection system captures the data and transmits the data to a server, via a network, (or a local computer) for analysis, record creation and record storage.

There has thus been outlined, rather broadly, some of the embodiments of the present disclosure in order that the detailed description thereof may be better understood, and in order that the present contribution to the art may be better appreciated. There are additional embodiments of that will be described hereinafter and that will form the subject matter of the claims appended hereto. In this respect, before explaining at least one embodiment in detail, it is to be understood that the various embodiments are not limited in its application to the details of construction or to the arrangements of the components set forth in the following description or illustrated in the drawings. Also, it is to be understood that the phraseology and terminology employed herein are for the purpose of the description and should not be regarded as limiting.

To better understand the nature and advantages of the present disclosure, reference should be made to the following description and the accompanying figures. It is to be understood, however, that each of the figures is provided for the purpose of illustration only and is not intended as a definition of the limits of the scope of the present disclosure. Also, as a general rule, and unless it is evidence to the contrary from the description, where elements in different figures use identical reference numbers, the elements are generally either identical or at least similar in function or purpose.

### BRIEF DESCRIPTION OF THE DRAWINGS

FIG. 1 is a block diagram of a tire defect detection system, according various aspects of the present disclosure.

FIG. 2 is a top view of the sections of the tire.

FIG. 3 is a perspective view of a first example embodiment of the tire defect detection system.

FIG. 4 is a perspective view of a second example embodiment of the tire defect detection system.

FIG. 5 is a perspective view of a third example embodiment of the tire defect detection system.

FIG. 6 is a top view of a fourth example embodiment of the tire defect detection system.

FIG. 7 is a diagram of an example data structure for storing the captured data.

FIG. 8 is a diagram of an example data structure for storing the defect data.

FIG. 9 is a diagram of an example data structure for the infrared data captured in the reference frame or the subsequent frame.

FIG. 10 is a diagram of the data structure for the reference frame or the subsequent frame physically relates to a section of the tire.

FIG. 11 is a diagram of an example data structure for the data of a difference frame.

FIG. 12 is a flow chart of an example method for frame capture.

FIG. 13 is a flow chart of a first example method for inspecting a tire and identifying defects.

FIG. 14 is a swim lane diagram of a second example method for inspecting a tire and identifying defects.

FIG. 15 is an example embodiment of pseudocode for subtracting the subsequent frame from the reference frame.

FIG. 16 is a diagram of an example image of the reference frame.

FIG. 17 is a diagram of an example image of the subsequent frame that includes a defect.

FIG. 18 is a diagram of an example image of the difference frame that includes the defect.

FIG. 19 illustrates a process of tire defect detection on a wheel balancer.

FIG. 20 illustrates a process of tire defect detection within a tire cage.

### DETAILED DESCRIPTION

### A. Overview

The tire defect detection system (e.g., system, inspection system, system for inspecting a tire), according to various aspects of the present disclosure, inspects a tire to identify defects in the tire without applying a wetting agent (e.g., water, liquid solution, soap) to the tire and without requiring an operator (e.g., user) trained in tire defect detection. The system may be fully automated or the system may allow some intervention by the operator; however, the ability and accuracy of the system in detecting defects does not depend on the skill, training or expertise of the operator.

Some example defects that can be detected using the systems and methods described herein include, but are not limited to, a tire leak, which is a common defect that can compromise the integrity and safety of a tire. This type of defect occurs when there is a puncture, crack, or other form of damage to the tire's rubber structure, allowing air to escape. Leaks can result from sharp objects such as nails or glass, wear and tear over time, or manufacturing defects. Detecting and addressing tire leaks is crucial to maintaining proper tire pressure, which ensures optimal handling, fuel efficiency, and safety. In the inspection process, thermal imaging and blob detection techniques can identify localized cooling caused by escaping air, pinpointing the exact location of the leak.

Another example defect involves leaks occurring at the interface between the tire and the wheel rim. This type of defect can arise from improper tire installation, damage to the rim, or degradation of the tire bead that seals the tire to the rim. Such leaks can be particularly problematic because they may not be immediately visible and can lead to gradual loss of air pressure. Ensuring a proper seal between the tire and rim is essential for maintaining tire pressure and preventing blowouts. During inspection, thermal imaging can reveal temperature changes at the rim interface, indicating potential leaks and allowing for timely repairs.

Leaks at the valve stem or Tire Pressure Monitoring System (TPMS) sensor are also significant defects that need to be addressed. The valve stem, which is responsible for allowing air to enter and exit the tire, can develop leaks due to wear, damage, or improper installation. Similarly, the TPMS sensor, which monitors tire pressure, can be a source of leaks if it becomes damaged or malfunctions. These leaks can lead to underinflated tires, affecting vehicle performance and safety. Inspection methods utilizing thermal imaging can detect temperature variations around the valve stem and TPMS sensor, highlighting leaks and ensuring that these components are functioning correctly.

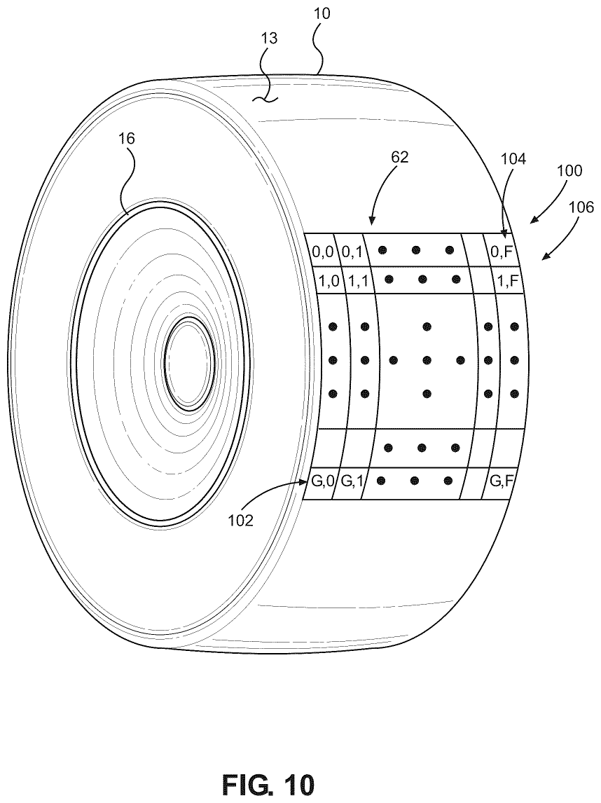

The tire defect detection system uses thermal (e.g., infrared) imaging and the analysis of thermal images to detect defects in the tire. An infrared camera is used to capture a thermal image, also referred to herein as a frame, of a section of the tire. The area of the frame, and hence the field-of-view of the infrared camera, is divided into rows and columns of pixels (e.g., data). Each pixel of the field-of-view falls on a respective portion of the section of the tire, as best seen in FIG. 10. When the infrared camera captures the thermal image of the section, each pixel of the frame captures (e.g., senses, records) the temperature of its corresponding portion of the section of the tire. For example, the field-of-view of the infrared camera may have 1024 pixels across and 512 pixels down for a total of 524,288 pixels. Each pixel relates to a specific portion (e.g., one of 524,288 distinct portions) of the section of the tire, so the value of each pixel in a frame represents the temperature of that specific portion of the section of the tire.

The system is adapted to detect defects that result in a loss of heated or cooled air (e.g., gas) from the tire, as provided by a thermal modulator that can either heat or cool the tire or the air pumped into the tire. For example, the thermal modulator can be used to heat the tire material. Air escaping from the tire via the defect can be detected through thermal imaging as the escaping air cools the tire material. In another example, the tire material is cooled while the air pumped into the tire is heated. Escaping air can be detected via thermal imaging. In yet another example, the tire material is not heated or cooled, but the air pumped into the tire can be heated or cooled, and escaping air can be detected via thermal imaging. It may be advantageous to use cooled air when ambient temperatures are high and heated air when ambient temperatures are low.

As the heated or cooled air from inside the tire exits a defect, the camera captures a frame. The system includes one or more infrared cameras that capture one or a series of frames of the section of the tire. The system compares pixels within the frame via blob detection to determine whether a portion of the section of the tire has increased or decreased in temperature. If a portion of the tire has heated or cooled, the difference in temperature is an indication that a defect exists in that portion of the section. Further, since the pixels of the frames relate to respective portions of the section of the tire, the pixels that show the difference in temperature relate to the location of the defect in the section of the tire. In other words, analysis of frame data identifies the physical location of the defect in the section of the tire.

The tire defect detection system may create and store records relating to the inspection of the tire and the defects found in the tire. The information created and stored may include frame data, thermal images, photographic descriptions, written descriptions and the results of analyzing some or all of the data captured by the system, and in particular the reference frame and the subsequent frame. The creation and storing of records may be fully automated.

The pixels of a frame may be presented as an image on a display. The warmer or cooler parts of the section of the tire, and therefore the defects, may be easily visible in the image.

The system may be implemented in various embodiments that range from a fully automated system that requires no user participation except to load and unload the tire from the system to a system that requires the user to position the tire and the infrared camera. Example embodiment of various systems are provided herein. The system may use a variety of techniques to analyze the thermal images to detect defects. Example techniques are provided herein.

### B. Some Possible Components of a System

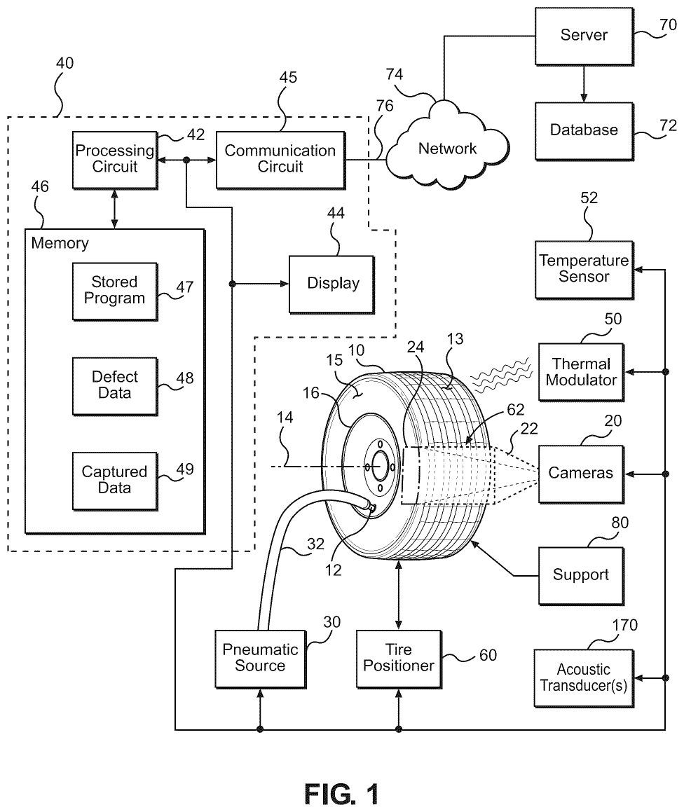

Prior to discussing example embodiments of the tire defect detection system, it is instructive to identify the various component that may be part of an embodiment. As best shown in FIG. 1, an embodiment of the system for inspecting the tire may include a support 80, one or more infrared cameras 20, a pneumatic source 30, a computing device 40, a thermal modulator 50, a temperature sensor 52, a tire positioner 60 and a server 70. An example embodiment may use a network 74 to accomplish communication between the computing device 40 and the server 70. In some instances, the system can incorporate one or more acoustic transducers 170, the use of which will be described in greater detail herein. It will be understood that while some embodiments contemplate the use of a server 70, the processes described herein can be executed locally at the computing device 40 level. Any process disclosed as being performed at the server level can be executed on the computing device 40 or another standalone computing device located remotely and in short or long range wireless, or wired communications with the computing device 40.

Some embodiments of the system will include more of the above components than other embodiments of the system. However, all embodiments of the system include one or more infrared cameras 20, the pneumatic source 30 and the computing device 40, or at least the processing circuit 42 of the computing device 40. The component discussed above are described in further detail below.

The term tire refers to the rubber portion of tire alone and/or the tire while mounted on a tire rim. While the term rim is used herein, it will be understood that the rim includes the metal or composite body to which the rubber tire is mounted. In some instances, the rim is also referred to as a wheel. The inspection of the tire occurs primarily on the rubber portion of the tire alone since that is the most likely place for a defect to occur. However, the valve stem 12 of the tire 10 is positioned on the rim of the tire 10 and may also be the source of a leak of air, so the rim, or a portion thereof, in particular the valve stem 12, may also be inspected to detect a defect.

### C. Sections of a Tire

The term “section of the tire” as used herein refers to a portion of the tire 10. The section 62 of the tire 10 includes the sidewall 15 (e.g., front, back, portion in field-of-view 24) of the tire or the tread 13 (e.g., portion within field-of-view 22) of the tire 10. Since the tire 10 is circular in shape, the sidewalls 15 or the tread 13 of the tire 10 extend around a 360-degree arc. The term “section” refers to any portion of the 360-degree circumference of the tire 10.

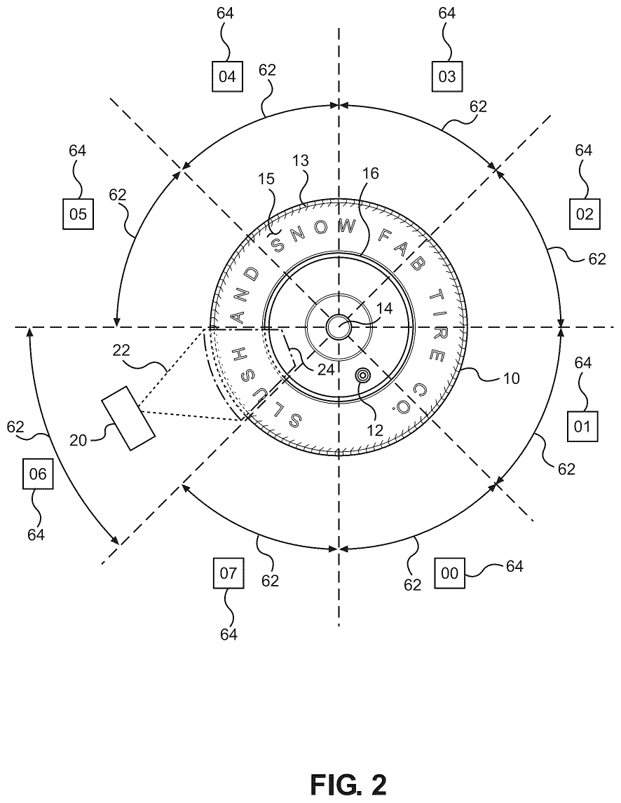

An example embodiment of the sections 62 of the tire 10 is shown in FIG. 2. The tire 10 shown in FIG. 2 is divided into eight sections 62, so each section 62 is a 45-degree portion of the circumference of the tire 10. Generally, the size of the section 62 corresponds to the size of the field-of-view (e.g., 22, 24) of the infrared camera 20. For example, as shown in FIG. 2, the field-of-view 22 and 24 of the infrared camera 20 covers a 45-degree section of the circumference of the tread 13 or the sidewall 15 of the tire 10. In another example, the field-of-view 22 and 24 of the infrared camera 20 covers a 36-degree portion of the circumference of the tire 10, so the section 62 of the tire 10 is 1/10 of the circumference of the tire, which means that the tire is divided into ten sections 62.

One issue that arises with respect to the sections 62 is accurately identifying the sections 62 after removal of the tire 10 from the test system. Further, during inspection of the tire, data is captured for one or more sections 62 of the tire 10. After testing, there needs to be away to correlate the captured data to the physical section of the tire 10. One example method to accurately identify the sections 62 of the tire 10, is to identify an origin section and location of the other sections with respect to the origin section.

The origin section of the tire 10 may be identified in a variety of ways. For example, the origin section of the tire 10 may be specified as the section that contains the valve stem 12 at the center of the section. Information regarding the origin section may be stored as origin indicia 65. In another example, the origin section may be described with respect to the writing on the sidewall 15 of the tire 10. For example, the letter “C” from “CO.” of the phrase “SLUSH AND SNOW FAB TIRE CO” may be used to identify the origin section. The origin indicia 65 may include a written description that identifies the letter “C.” as appearing in a specific portion (e.g., edge) of the section. The origin indicia 65 may include a photograph of the sidewall of the section showing the position of the letter “C.” with respect to the boundaries of the section.

All of the sections 62 of the tire 10, including the origin section, may be identified using a section indicia 64. For example, as shown in FIG. 2, the origin section is assigned the section number 00, the next section 62 is assigned the section number 01, the next section 62 is assigned the number 02, and so forth. Information may be stored that describes the direction of rotation 67 to move from one section 62 to another section 62. For example, to go from the section 62 identified as 00 to the section 62 identified as 01, the tire is rotated in the clockwise direction. The origin indicia 65, the section indicia 64 for each section, the direction of rotation 67 may be stored in captured data 49.

The origin indicia 65, the section indicia 64 and the direction of rotation 67 may be used as follows. Assume that the tire 10 is inspected and defects are identified. Assume that all captured and calculated data are stored in captured data 49 and/or defect data 48. Further assume the tire is sent to the manufacturer for failure analysis. The manufacture may use the origin indicia 65 to accurately identify the origin section of the tire 10. The manufacturer may use the section indicia 64 and the direction of rotation 67 to rotate the tire from the origin section to any section 62 that has a defect 164. The origin indicia 65, the section indicia 64 and the direction of rotation 67 make it easy to identify the physical portion of the tire to which the captured data 49 and/or the defect data 48 relates.

Finding or establishing an origin section may be omitted if the user manually marks the defect identified as the section-under-test (e.g., section within field-of-view) before moving to the next section for inspection. Manually marking may include using some form of marking pen to draw a circle around the defect. The section may be presented on the display 44 so that the user may see the location of the defect in the section-under-test, so the user may mark the defect.

### D. Thermal Defect Detection

The tire 10, is inflated with heated or cooled air, retains air (e.g., gas) inside at a certain pressure (e.g., pounds per square inch, PSI). The pressure of the air inside the tire is greater than the atmospheric pressure, so when the tire 10 develops a defect 164 (e.g., hole, puncture, flaw), the air inside the tire exits the tire through the defect 164. The rate at which the air exits the flaw depends on the size and type of the defect 164. A large diameter puncture with clean edges allows the air to escape quickly and at a high rate thereby deflating the tire 10 quickly. A small diameter puncture with overlapping edges that obstruct the puncture or a flaw between the layers of the tire 10 inhibits the escape of air so the air escapes more slowly and at a lower rate.

From the perspective of the field-of-view of the infrared camera 20, the field-of-view (e.g., 22, 24) covers a section 62 of the tire 10. As best shown in FIG. 17, the escaping air from a defect 164 located in the section heats or cools the portion (e.g., row 635 and column 1271 to row 852 and column 1546, (635, 1271) to (852, 1546)) of the section around the defect 164, and not the section as a whole (e.g., (0,0) to (G, F)). So, the area of the tire 10 that is heated or cooled by the air escaping from the defect 164 is limited to the portion of the section 62 that is proximate to (e.g., around) the defect 164. The localized heating or cooling of the tire 10 due to the escape of air from the defect 164 enables the infrared camera 20 to detect a change of temperature in a portion (e.g., (635, 1271) to (852, 1546)) of the section 62 and thereby identify the defect 164 in that section 62 of the tire 10. In other words, the decrease in the temperature of the portion of the section 62 occurs as a result of a flow of air out of the tire through the defect 164 thereby heating or cooling the portion of the section 62.

### E. Support

The support 80 is adapted to support the tire 10. The support 80 may support the weight of the tire 10 in whole or in part. The support 80 cooperates with the tire positioner 60 to position (e.g., manipulate, rotate) the tire 10. The support 80 may include a tire spindle (e.g., post) 82 that is positioned (e.g., inserted) through the center of the rim of the tire 10 to support the weight of the tire 10 and to permit the tire 10 to be rotated around the central axis 14 of the tire spindle 82. The support 80 may also include a component spindle 84 for supporting components of the inspection system. The support 80 may further include a base 86 for stabilizing and supporting the support 80 and the component of the system.

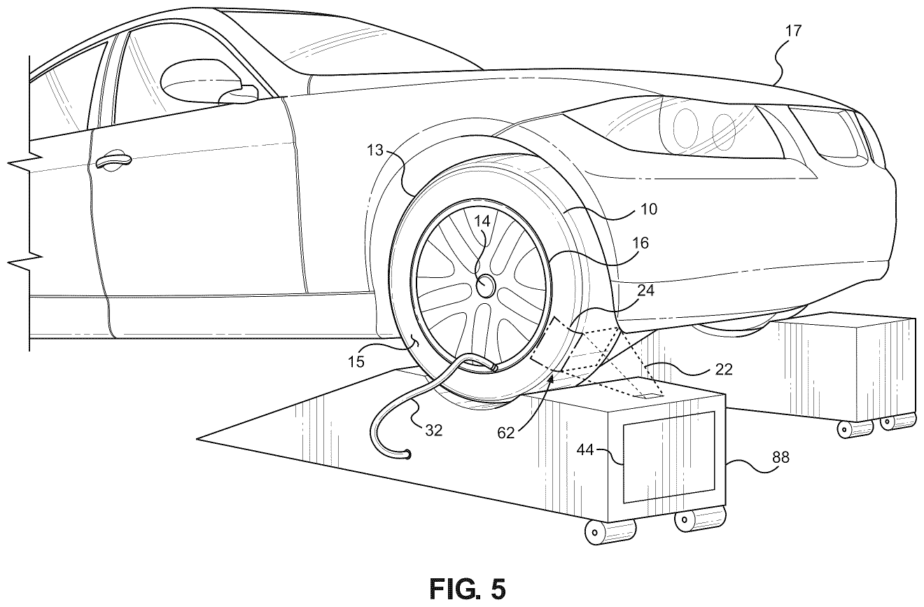

In another example embodiment, as best shown in FIG. 5, ramp support 88 is shaped in part like a ramp to allow the vehicle 17 to drive the tire 10 onto the ramp support 88 for inspection. The ramp support 88 may support the weight of the tire 10 and a part of the weight of the vehicle 17 because the tire 10 remains attached to the vehicle 17 during examination.

### F. Pneumatic Source

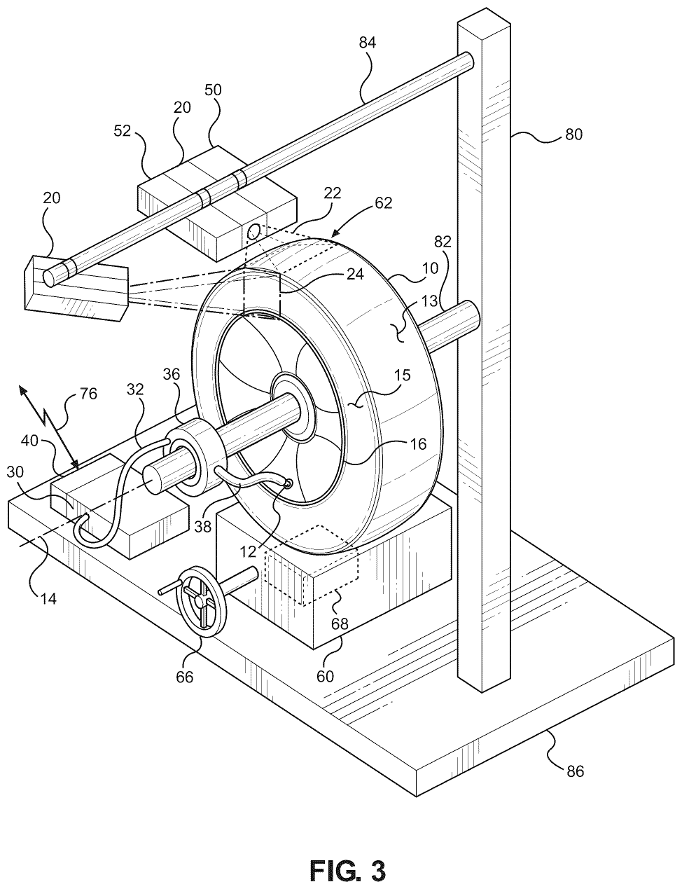

The pneumatic source 30 is adapted to inflate the tire 10. As best seen in FIGS. 3-6, the pneumatic source 30 may include a hose 32. The hose 32 is adapted to connect to the valve stem 12 of the tire 10, so the pneumatic source 30 inflates the tire 10 via the hose 32. In an example embodiment, the computing device 40 controls the operation of the pneumatic source 30 to inflate the tire 10.

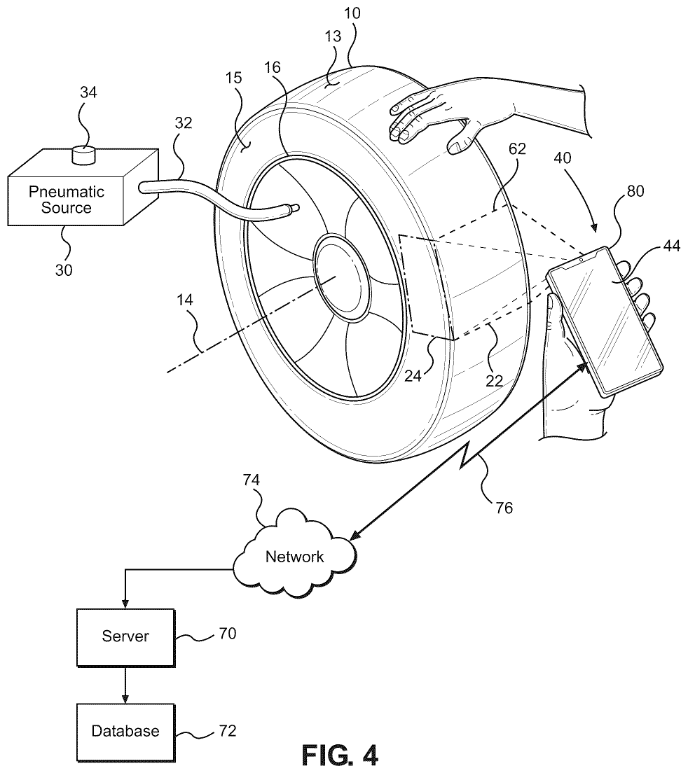

In another example embodiment, as best seen in FIG. 4, the pneumatic source 30 is operated manually by a user to inflate the tire 10. In an example embodiment, the pneumatic source 30 inflates the tire 10 to a pre-determined pressure. In an example embodiment, the pneumatic source 30 inflates the tire 10 to the recommended pressure (e.g., 30 PSI) for the tire 10. The pneumatic source 30 may include any type of air compressor. The hose 32 may connect to the valve stem 12 in any manner. The hose 32 may connect to a device (e.g., pneumatic collar 36) that delivers air to the valve stem 12 of the tire 10 while the tire 10 rotates.

The computing device 40, and in particular a processing circuit 42 of the computing device 40, may be adapted to control the pneumatic source 30. The processing circuit 42 may be adapted to control the pneumatic source 30 responsive to executing a stored program. The processing circuit 42 may instruct the pneumatic source 30 to provide air to (e.g., inflate) the tire 10. The processing circuit 42 may instruct the pneumatic source 30 to cease providing air to the tire 10. The processing circuit 42 may receive information regarding the pressure of the tire 10, so the processing circuit 42 may cease providing air to the tire 10 when the tire 10 reaches a predetermined pressure.

The pneumatic source 30 may measure (e.g., detect) a pressure of the air in the tire 10. The pneumatic source 30 may report the pressure of the air in the tire 10 to the processing circuit 42. The processing circuit 42 may instruct the pneumatic source 30 cease providing air to the tire 10 when the tire 10 reaches a predetermined pressure.

### G. Tire Positioner

The tire positioner 60 is adapted to position the tire 10. The tire positioner 60 is adapted to position one section 62 of the tire 10 in the field-of-view (e.g., 22, 24) of the infrared camera 20. The tire positioner 60 may successively position each section 62 of the tire 10 in in the field-of-view of the infrared camera 20.

The tire positioner 60 may identify each section 62 of the tire 10. The tire positioner 60 may uniquely identify each section 62 of the tire 10. The data used to identify the section 62 of the tire 10 is referred to as the section indicia (e.g., identifier) 64. The section indicia 64 may be used to identify the section 62 of the tire 10 presently positioned in the field-of-view (e.g., 22, 24) of the infrared camera 20. The tire positioner 60 may report the section indicia 64. The section indicia 64 may be recorded (e.g., stored). The section indicia 64 may be associated with the data (e.g., frames) captured with respect to the section 62. In an example embodiment, a rotary encoder 68 is used to identify each section 62 of the tire 10. The section indicia 64 may be related to an origin section of the tire 10.

The tire positioner 60 may identify one section 62 of the tire 10 as being an origin section. The origin section of the tire 10 is one section 62 that may be used to reference the other sections 62 of the tire 10. Information regarding the origin section may be recorded, so the origin section, and hence all other sections, may be identified after testing is complete. For example, as discussed above, the origin section of the tire 10 may be the section of the tire 10 that includes the valve stem 12 at a specific location (e.g., left edge, right edge, middle) in the section 62. The origin section of the tire 10 may be the section 62 that includes the tire specification information printed on a sidewall of the tire 10 at a specific location in the section 62. A photograph, not thermal, of the sidewall 15 of the origin section may be captured to later identify the origin section. The photograph may be stored along with any other data (e.g., captured data 49) associated with the tire 10.

For example, assume that during inspection the tire 10, the origin section of the tire is identified as the section in which the valve stem 12 is in the center of the section 62. Each section 62 is ⅛ of the circumference of the tire 10, such that each section 62 is a 45-degree portion of the circumference of the tire 10. Further, during inspection a rotary encoder 68 is used to provide section indicia 64 for each of the sections 62. The section indicia 64 for the origin section is the number 00, the section indicia 64 for the next section, as the tire 10 rotates clockwise from the origin section, is 01, the section indicia 64 for the next section 62 in a clockwise direction is 02, and so forth. Assume that the information needed to identify the origin section, the section size, and the section identifier for all of the sections is recorded along with the frames captured during the inspection of the tire 10. Assume that during inspection, the defect 164 is identified in section 04 and that the tire is returned to the manufacturer for failure analysis.

Upon receipt of the tire, the manufacture may use information regarding the origin section of the tire 10 to position the tire 10 on a support with the origin section positioned in the field-of-view of the infrared camera 20. Having identified the origin section, the manufacturer may use the section indicia 64 of the location of the defect 164 (e.g., section 04) to rotate the tire to where the defect 164 is located. Now that the section 62 of the defect 164 has been properly identified, the manufacturer may begin further analysis of the defect 164. Lacking information regarding the origin section of the tire 10, the manufacturer could not accurately and quickly identify the section 62 where the defect 164 is located.

In an example embodiment, the tire positioner 60 automatically locates and/or identifies the origin section. An example embodiment the tire positioner 60 automatically rotates the tire 10 from one section 62 to any other section 62. The tire positioner 60 may provide data regarding the sections 62 of the tire 10 for storing with and relating to other data captured and/or calculated with respect to the sections 62 of the tire 10.

The computing device 40, and in particular a processing circuit 42 of the computing device 40, may be adapted to control the tire positioner 60. The processing circuit 42 may be adapted to control the tire positioner 60 responsive to executing a stored program. The processing circuit 42 may instruct the tire positioner 60 to position a section 62 of the tire 10 in the field-of-view (e.g., 22, 24) of the infrared camera 20.

The tire positioner 60 may be integrated with the support 80. The tire positioner 60 may be integrated with any type of equipment used to maintain tires. For example, the tire 23 positioner 60 may be integrated with a tired jack and a tire changer machine, a tire balancing 24 machine and a tire clamping machine.

### H. Thermal Modulator

The tire defect detection system may further include a thermal modulator 50 adapted to heat or cool the tire 10 or rim. Examples of thermal modulators that can be used in this tire defect detection system include infrared heaters, hot air blowers, and cooling systems-just to name a few. Infrared heaters emit radiation that directly heats the tire surface, ensuring uniform temperature elevation before and during the inflation process. Hot air blowers can be employed to heat the air blown into or onto the tire, effectively increasing the temperature or the air or tire itself. Conversely, cooling systems such as refrigerant-based coolers or cold air blowers can be used to lower the tire's temperature. These modulators help create a temperature differential that accentuates defects, making them more detectable by thermal imaging and blob detection techniques.

The thermal modulator 50 may heat or cool the tire 10 prior to and/or while inflating the tire 10. Heating or cooling the tire 10 accentuates the decrease or increase in the temperature that results from an escape of air from the defect 164 in the tire 10. Heating or cooling the tire 10 makes it easier to identify defects 164 in the tire 10 because it increases the difference in temperature between the portion of the section 62 cooled by the escaping air and the remainder of the section 62 of the tire 10.

The thermal modulator 50 may heat or cool the tire 10 in any method. The thermal modulator 50 may heat by conduction, convection, radiation, coolant, and/or refrigerant. The thermal modulator 50 may include an infrared heater. The thermal modulator 50 may include a space heater. The cooling source may include blowing cool air or radiant cooling via coolant and/or refrigerant.

Another approach for accentuating the difference in the temperature that results from the escape of the gas from the defect 164 is to inflate the tire 10 with a gas whose temperature is more or less, possibly significantly more or less, than the temperature of the tire. The warmer or cooler initial temperature of the gas in combination with the warmed or cooled air will cause an increased temperature difference between the area around the defect 164 and the remainder of the tire 10.

In an example embodiment, the thermal modulator 50 heats the tire 10 prior to the infrared camera 20 capturing the reference frame 100. In an example embodiment, the thermal modulator 50 heats the tire 10 while pneumatic source 30 inflates the tire 10. In another example embodiment, the thermal modulator 50 heats the tire 10 while the tire is deflated. In an example embodiment, the thermal modulator 50 heats only the section 62 of the tire positioned in the field-of-view (e.g., 22, 24) of the infrared camera 20. In another example embodiment, the thermal modulator 50 heats the entire tire.

The computing device 40, and in particular a processing circuit 42 of the computing device 40, may be adapted to control the thermal modulator 50. The processing circuit 42 may be adapted to control the thermal modulator 50 responsive to executing a stored program. The processing circuit 42 may instruct the thermal modulator 50 to begin heating the tire 10. The processing circuit 42 may instruct the thermal modulator 50 to cease heating the tire 10.

### I. Temperature Sensor

The tire defect detecting system may include the temperature sensor 52. The temperature sensor 52 may detect the temperature of the tire 10 in general and/or the pneumatic heated or cooled air. The temperature sensor 52 is not used to detect the respective temperatures of the various portions of the section 62 of the tire 10, as is performed by the infrared camera 20. The temperature sensor 52 may cooperate with the processing circuit 42 and the thermal modulator 50 to detect the temperature of the tire 10, so the thermal modulator 50 may continue heating or cease heating the tire 10. In some instances, multiple temperature sensors can be incorporated. For example, one temperature sensor can be used to detect temperature differences relative to a tire surface, while another temperature sensor can be used to determine the temperature of the air provided by the pneumatic source 30

### J. Acoustic Transducer

The tire defect detection system is equipped with components designed to identify tire defects through a combination of acoustic and thermal imaging techniques. In one example, acoustic transducers 170 are strategically placed around the tire 10 to emit and receive high-frequency sound waves. These acoustic transducers 170 are used to detect changes in wave propagation caused by defects such as air pockets, cracks, or separations within the tire 10. For example, an acoustic transducer can include an ultrasonic transducer. When ultrasonic waves emitted by the ultrasonic transducer pass through the tire 10, any anomalies or irregularities in the tire's structure affect the waves' behavior. The transducers 170 capture these changes, and the data is sent to the computing device 40. Other acoustic transducers or sensors can be used, as would be known to one of ordinary skill in the art.

The computing device 40 processes the signals from the transducers 170 using analytical techniques like time-of-flight analysis and frequency response analysis. These methods help in accurately identifying and locating defects within the tire 10. The time-of-flight analysis measures the time it takes for the ultrasonic waves to travel through the tire 10 and back to the transducers 170, while frequency response analysis examines how the defects alter the frequencies of the waves. By analyzing these factors, the system can detect even minor defects that might not be visible through traditional inspection methods.

In addition to the acoustic detection, the system incorporates thermal imaging to enhance defect visibility. The pneumatic component 30 of the system inflates the tire 10 with either hot or cold air (heated or cooled by the thermal modulator 50), creating a temperature differential that makes defects more apparent. As air escapes through any defects, it causes localized cooling or heating, which is captured by thermal imaging cameras 20. These cameras 20 provide a visual representation of the tire's thermal profile, highlighting areas with potential defects.

Temperature sensors 52 are included to monitor the tire's temperature throughout the inspection process, ensuring that the conditions are optimal for both ultrasonic and thermal detection methods. The data collected by the sensors 52, along with the thermal images and ultrasonic signals, are processed and analyzed by the computing device 40. The data captured by the sensors 52 can be used to determine defects such as when an air leak due to a tire puncture produces a cooler thermal signature relative to a heated tire surface. That is, air escaping from the defect will create a locally “cooler” area relative to the heated area of the tire surface that surrounds the leak.

The system also includes data storage 46 and display 44 components, which record the inspection results and provide a comprehensive visualization of the tire's condition. The display 44 allows operators to view real-time feedback and detailed analysis of the detected defects, making it easier to identify and address any issues.

A user-friendly interface is provided to facilitate the operation of the system. This interface allows users to control the inspection process, adjust settings, and review inspection results. The integration of these components ensures a thorough and efficient tire inspection process, capable of detecting even the smallest defects that could compromise tire performance and safety.

### K. Cameras

The tire defect detecting system includes one or more infrared cameras 20. As discussed above the infrared camera 20 includes a field-of-view. When the field-of-view of the infrared camera 20 is positioned over the tread 13, the field-of-view is referred to as the field-of-view 22, whereas, when the field-of-view is positioned over the sidewall 15, is referred to as field-of-view 24. The infrared camera 20 may be positioned over the tread 13 to capture frame data (e.g., reference frame 100, subsequent frame 106) within the field-of-view 22 then repositioned over the sidewall 15 to capture frame data within the field-of-view 24 or a first infrared camera 20 may be positioned to capture frame data for field-of-view 22 and a second infrared camera 20 positioned to capture frame data for the field-of-view 24.

The infrared camera 20 captures temperature data regarding the section 62 of the tire 10 within the field-of-view (e.g., 22, 24) by sensing the temperature of the section 62 of the tire 10. As discussed above, the infrared camera 20 captures a frame of data in which each pixel of the frame represents the temperature of a respective portion of the section of the tire 10.

As discussed above, the frame data captured by the infrared camera 20 is used to detect a decrease in the temperature of a portion of the section 62 of the tire 10 that results from an escape of air through a defect 164 in the tire 10.

Any type of blob detection subtraction (e.g., pixel-wise, cluster, column-wise, row-wise, run-length encoded) may be performed to determine the difference. As disclosed above, Blob detection is an image processing technique that can be effectively utilized to identify and analyze regions in an image that differ in properties such as brightness or color from their surroundings. In the context of tire defect detection, blob detection can be applied to thermal (infrared) images to pinpoint areas where localized cooling indicates a defect.

When a tire is inflated with heated or cooled air, any defects such as punctures or cracks will allow air to escape, causing a temperature change in the affected area. The tire defect detection system captures high-resolution thermal images of the tire, either while it is mounted on the vehicle or after removal. The computing device 40 analyzes these images to detect blobs, which are regions that deviate from the normal temperature pattern of the tire.

The process involves comparing a reference thermal image captured shortly after inflation to subsequent images taken after a period of time. Blob detection algorithms identify regions where the temperature has significantly decreased, suggesting an escape of air. The detected blobs are then further examined to assess the nature and severity of the defects, enabling precise identification and localization within the tire. This method offers a dry, automated alternative to traditional manual inspection techniques, reducing the need for trained technicians and hazardous waste disposal.

### L. Captured Data

The data captured or determined by the tire defect detecting system, herein referred to as captured data 49, may be stored. Data may be captured and/or determined by any component of the tire defect detecting system. Data may be captured and/or determined for each section of the tire 10.

For example, captured data 49 may include the tire identifier 11, the origin indicia 65, the number of sections 63, the direction of rotation 67, and the section record 90. The tire identifier 11 may include a serial number. The serial number may include the DOT serial number found on tires made in the United States. The tire identifier 11 may include a written or photographic description of the tire 10.

The origin indicia 65 includes information that describes the origin section of the tire 10. As discussed above, the origin section is the section of the tire used to reference the other section of the tire. The origin indicia 65 may include a standard feature of the tire (e.g., valve stem 12), a written description and/or a photograph of the origin section.

The number of sections 63 stores the number of sections into which the tire 10 was divided for inspection. The data for each section 62 is stored in a respective section record 90, so the number of records identified as section record 90 corresponds to the number of sections 63. The number of sections 63 may be a number that is used to divide the circumference of the tire to identify the size of each section. The number of sections 63 may be identified by identifying the number of degrees of a complete circle used for each section. For example, storing the number eight for the number of sections 63 provides information that the tire 10 was divided into eight sections for inspection and that each section is 45 degrees of the circumference of the tire. Storing the number 10 degrees for the number of sections 63 provides the information that each section of the tire 10 is 10 degrees of the circumference of the tire which means that there are 36 sections for the tire 10.

The direction of rotation 67 identifies the direction (e.g., counterclockwise, clockwise) that the tire 10 was rotated from the origin section to the next section 62 when the sections exceed two are considered in order. The origin indicia 65, the number of section 63 and the direction of rotation 67 make it possible to positively identify and to accurately position each section 62 of the tire 10 after initial inspection of the tire 10.

Section record 90 includes the section indicia 64, the reference frame 100 and the subsequent frame 106. As discussed above, the infrared camera 20 may capture a plurality of subsequent frames, so the subsequent frame 106 of the section record 90 may include the plurality of subsequent frames 106. The section record 90 may further include the difference frame 108 (not shown).

The reference frame 100, the subsequent frame 106 and/or the difference frame 108 may be used to identify defects in the section 62 of the tire 10 during examination or after examination. Captured data 49 may be transmitted (e.g., sent) to a server 70.

### M. Computing Device

In an example embodiment, the tire defect detection system includes the computing device 40. The computing device 40, and thereby the tire defect detection system, includes a processing circuit 42, a display 44, a communication circuit 45 and a memory 46. The computing device 40 may include the display 44. The memory 46 is adapted to store data. For example, the memory 46 is adapted to store at least one of a stored program 47, the defect data 48 and the captured data 49. The processing circuit 42 is adapted to control the memory 46. In particular, the processing circuit 42 is adapted to store data in and to retrieve data from the memory 46.

Any device capable of performing the functions of the computing device 40 may be the embodiment of the computing device 40. In an example embodiment of the tire defect detection system, a smart phone 41 is the embodiment of the computing device 40. In another example embodiment, a tablet is the embodiment of the computing device 40. In another example embodiment, a mobile computer is the embodiment of the computing device 40.

The communication circuit 45 is adapted to transmit or receive data. In an example embodiment, the processing circuit 42 is adapted to control the communication circuit 45. The processing circuit 42 is adapted to retrieve data from the memory 46 and to provide the data to the communication circuit 45 for transmission. The processing circuit 42 is adapted to receive data from the communication circuit 45 and to store the data in the memory 46. In an example embodiment, the communication circuit 45 transmits at least one of the defect data 48 and the captured data 49, which includes the reference frame 100 and the subsequent frame 106 for the respective section record 90, so the communication circuit 45 transmits at least one of the reference frame 100 and the subsequent frame 106.

### N. Network, Server, Database

The tire defect detection system may include network 74, server 70, and database 72. The computing device 40 may communicate (e.g., transmit to, received from) with the server 70 via the network 74. The computing device 40 may communicate with the network 74 using communication link 76. Communication link 76 may be a wired and/or a wireless communication link.

In an embodiment, the communication circuit 45 of the computing device 40 transmits at least one of a defect data 48, including the reference frame 100 and the subsequent frame 106 for one or more sections 62 of the tire 10 to the server 70. The server 70 stores at least one of the defect data 48, the reference frame 100 and the subsequent frame 106 for one or more sections 62 of the tire 10 in the database 72.

The server 70 may use the captured data 49 to determine whether any section 62 of the tire 10 has a defect 164. The server 70 may analyze the captured data 49 to determine whether any section of the tire 10 has a defect 164 after, including long after, the inspection of the tire 10 has been completed. The server 70 may store the captured data 49 in the database 72. The server 70 may store multiple versions of the captured data 49 for the same tire 10 in the database 72. The captured data 49 may include a date (e.g., date and time, date stamp) that the inspection was performed and the captured data 49 captured.

The database 72 may maintain a historical record of the inspections of the tire 10. The database 72 may include the captured data 49 captured during each inspection of the tire 10. The various captured data 49 may be distinguished by a timestamp.

### O. Identifying Defects

The tire defect detection system may use the data captured by the infrared camera 20 to determine whether a defect 164 exists in a section 62 of the tire 10. As discussed above, the defect 164 is detected in the section the two of the tire 10 by detecting a difference in the temperature of a portion of the section 62 of the tire 10. A variety of techniques may be used to detect whether a defect exists in the section 62 of the tire 10.

In an example embodiment, the system (e.g., processing circuit 42, server 70) compares the reference frame 100 to the subsequent frame 106 to detect a decrease in the temperature of a portion of the section. The decrease in the temperature occurs during the period of time between capture of the reference frame 100 and capture of the subsequent frame 106. The decrease in the temperature of the portion of the section indicates a defect. In example embodiment, comparing the reference frame 100 to the subsequent frame 106 comprises subtracting the subsequent frame 106 from the reference frame 100 to form a difference frame 108, as discussed above. The difference frame 108 is then analyzed to determine whether any defects 164 exist in the section 62.

Subtracting may be accomplished via the blob detection method within the same frame, where the algorithm identifies regions with significant temperature differences as potential defects. This method focuses on detecting blobs, or clusters of pixels, that exhibit a notable deviation from the surrounding area in the thermal images.

Subtracting may also the accomplished by combining proximate pixels in the reference frame into respective groups and subtracting the value of the groups from each other. For example, the values of four proximate pixels (e.g., (0,0), (0, 1), (1, 0), (1, 1)) may be combined (e.g., averaged, subtracted, added, squared, cubed, any function, any combination thereof) in the reference frame 100 and/or the subsequent frame 106 prior to subtracting. The four proximate pixels may be assigned the combined value for pixel-wise subtraction or the group value for the pixels of the subsequent frame 106 may be subtracted from the group value of the reference frame 100. The resulting values may be stored in the difference frame 108.

Analysis or further analysis may be performed on the difference frame 108, the reference frame 100, the subsequent frame 106 or any combination thereof to determine the presence of a defect 164 in the section 62. In an example embodiment, blob detection techniques are used to analyze the difference frame 108 to identify defects. In another example embodiment, blob detection techniques are used to analyze the subsequent frame 106 to identify defects. In an embodiment that captures a plurality of subsequent frames 106, blob detection techniques may be used to analyze one or more subsequent frames of the plurality, alone or in combination, to identify defects 164.

Blob detection includes any technique that detects (e.g., identifies) regions in a digital image (e.g., reference frame 100, subsequent frame 106, difference frame 108) that differ in properties. Properties may include brightness, color or in this case temperature. Blob detection compares the properties of one region (e.g., one or more pixels) of the image to surrounding regions of the image. A blob is a region (e.g., one or more adjacent pixels) in which one or more properties are the same or approximately the same. Blob detection May detect adjacent pixels (e.g., first region) that have similar properties then compare the properties of the first region to the properties of surrounding regions. In this case a group of pixels that have approximately the same temperature may be compared to other groups of pixels to determine whether there is a difference in temperature between the groups.

The most common method used for blob detection is convolution. The property of 7 interest (e.g., temperature) in image may be expressed as a function of its position in the image. The function may then be convoluted with another function (e.g., a different temperature-to-position function) to identify areas (e.g., blobs) of the image that have common properties. Blob detection techniques may be used to identify areas that have experienced a decrease in temperature, and are therefore likely defects 164, and the location of the defect 164 in the section the two of the tire 10.

Blob detection techniques may also include differential methods based on derivatives of a function with respect to position and/or local extrema methods based on finding local maxima and/or minima of the function. Once a blob (e.g., region) is identified, the region may be further processed to identify objects in the region. Blob extraction techniques used to identify objects in the blob may include connected-component labeling and connecting-component analysis. Edge detector techniques and corner detector techniques may be used to identify the characteristics of the blob. Blob extraction techniques may be used to identify a type (e.g., puncture, gash, layer flaw) of the defect 164, the severity (e.g., depth, width, height, area) of the defect 164.

In an example embodiment, comparing the reference frame 100 to the subsequent frame 106 to detect a decrease in the temperature of the portion of the section 62 includes subtracting the subsequent frame 106 from the reference frame 100 to form the difference frame 108 and analyzing the difference frame 108 using blob detection techniques to identify the portion (e.g., area) of the section 62 that experienced the decrease in the temperature. In another example embodiment, comparing the reference frame 100 to the subsequent frame 106 includes using blob detection techniques to identify a location of the defect 164. In another embodiment, comparing the reference frame 100 to the subsequent frame 106 includes using blob detection techniques to identify at least one of a type of the defect 164 and a severity of the defect 164. In another example embodiment, the processing circuit 42 compares the reference frame 100 to the subsequent frame 106 using blob detection techniques to detect the decrease in the temperature of the portion of the section 62 thereby detecting the defect 164. In another example embodiment, the processing circuit 42 compares the reference frame 100 to the subsequent frame 106 by subtracting the subsequent frame 106 from the reference frame 100 to form a difference frame 108 and analyzing the difference frame 108 using blob detection techniques to identify the portion of the section 62 that experienced the decrease in the temperature.

Other analysis techniques for identifying defects in a section 62 may include histogram analysis, texture analysis, feature identification techniques used in the field of computer vision. Feature identification techniques include edge detection, corner detection, interest point detection, region detection, ridge detection, edge direction detection, changing intensity detection, autocorrelation, motion detection, template matching and Hough transforms.

### P. Defect Data

Once a defect 164 has been identified, information regarding the defect 164 may be stored in defect data 48. The defect data 48 is a record of all the defects identified in a specific tire 10. The defect data 48 for different tires will be different. The defect data 48 may include a date to identify the date on which the tire 10 was inspected to find the defects 164 that are recorded in the defect data 48. A specific tire 10 may have multiple defect data 48 with different dates.

In an example embodiment, defect data 48 includes the tire identifier 11, the origin indicia 65, the number of sections 63, the direction of rotation 67, the number of rows in the frame 102 (e.g., reference frame 100, subsequent frame 106, difference frame 108), the number of columns in the frame 104, and one or more defect records 110. As discussed above with respect to the captured data 49, the tire identifier 11, the origin indicia 65, the number of sections 63 and the direction of rotation 67 may be used to positively identify and to accurately position each section 62 of the tire 10 after the initial inspection of the tire 10. The number of rows in the frame 102 and the number of columns in the frame 104 identify the number of rows and columns in the frame data (e.g., reference frame 100, subsequent frame 106, difference frame 108) used to identify the defect 164, so that the location of the defect 164 in the section 62 may be accurately located and identified.

The defect record 110 includes section indicia 64, the row number 116, the column number 118 and the area 119. The section indicia 64, as discussed above, identifies the section 62 of the tire 10 where the defect 164 is located. The row number 116 and column number 118 identify the row and column of the location of the defect with respect to the frame, and thereby the physical location of the defect 164 in the section indicia 64. The row and column numbers may refer to any portion of the defect (e.g., top, bottom, center). In an example embodiment, the row number 116 and column number 118 identify the pixel at the center of, or close to the center of the defect. The area 119 describes the area covered by the defect or the area covered by the defect 164 and the area of decreased temperature of the tire 10 around the defect 164. The area 119 may be described by the coordinates (e.g., (row number, column number)) of the corners of a geometric shape (e.g., square, rectangle) that encloses the defect and/or the area of decreased temperature of the tire 10. The area 119 may be described by the coordinates of the edges around the defect. The area 119 may be described in any manner that uses one or more coordinates to describe an area.

The defect data 48 may be used to accurately identify the location of a defect in the tire 10, so that the location of the defect may be found once again after the initial inspection has been completed, and possibly the tire shipped to another facility for further analysis and work.

### Q. Display

The tire defect detection system may include a display 44. The display may be a part of the computing device 40. The display 44 may be a part of the server 70. The display 44 may be standalone, but receive data for presentation from the computing device 40 and/or the server 70. The display 44 may be positioned in the support (e.g., 80, 88). The frames (e.g., reference frame 100, subsequent frame 106, difference frame 108) captured by the infrared camera 20 are infrared images of the tire. Being images, the frames may be presented on the display 44 for viewing by a user.

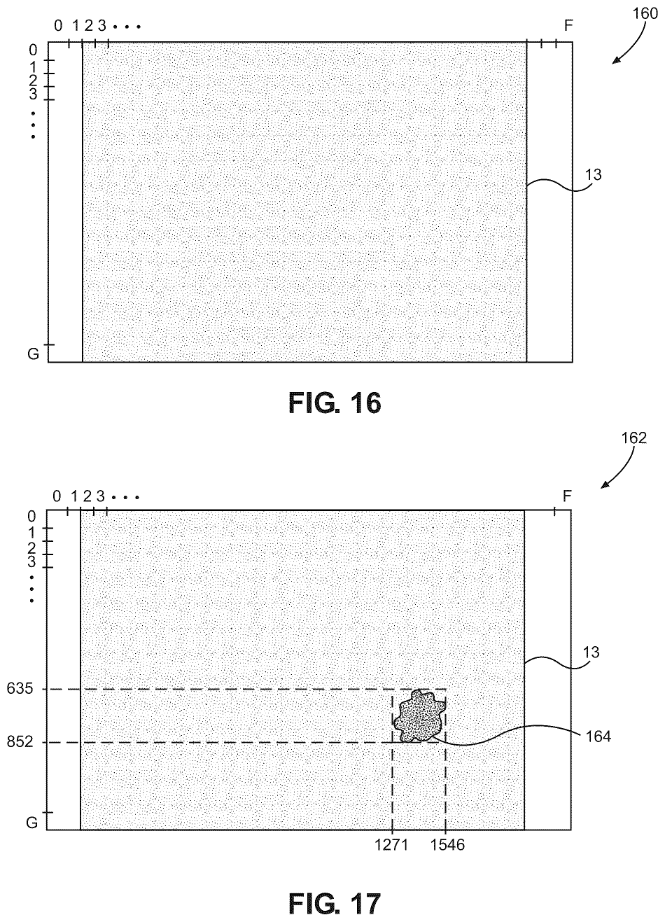

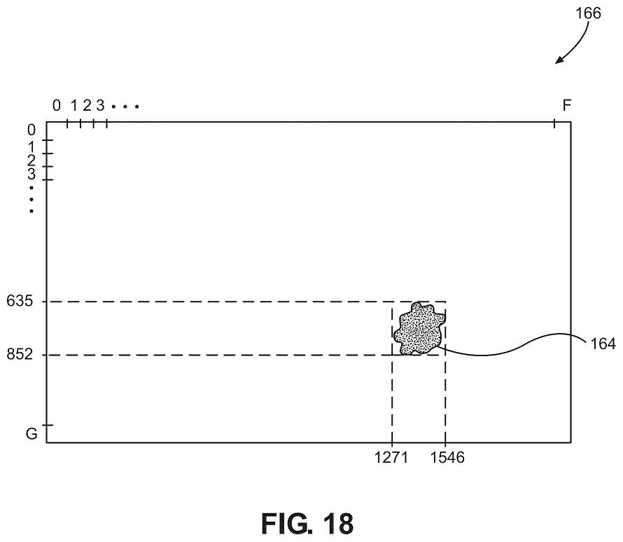

In an example implementation, the system for inspecting the tire includes the display 44, the display 44 is adapted to present the reference frame 100, the subsequent frame 106, and/or the difference frame 108 as an image, whereby the defect 164 is visible in the image. For example, referring to FIGS. 16-18, the display 44 may be adapted to display the reference frame 100 as the reference image 160, the subsequent frame 106 as the subsequent 14 image 162, and the difference frame 108 as difference image 166. Because the reference frame 100 is captured shortly or immediately after inflating the tire 10, the reference image 160 shows the temperature of the section 62 of the tread 13 of the tire 10 as being substantially the same across the reference image 160. After a period of time, air has escaped through defect 164 thereby cooling the area of the tread 13 of the tire 10 around the defect 164, so the subsequent image 162 shows an increased difference in the temperature between the area around the defect and the rest of the section 62.

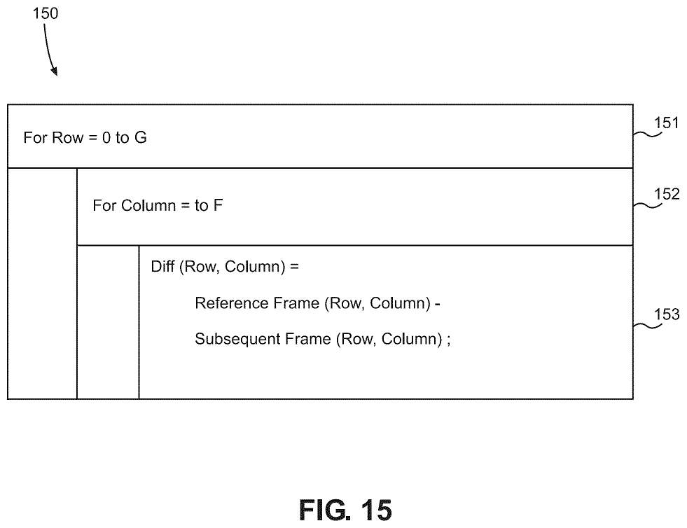

The display 44 may also be adapted to present the difference frame 108 or any other type of frame after analysis has been performed to identify the defect 164. For example, FIG. shows the presentation of the difference image 166 on the display 44. In this example, the difference frame 108 was created by subtracting the subsequent frame 106 from the reference frame 100. Since the temperature of the tread 13 in both the reference frame 100 in the subsequent frame 106 are essentially the same across much of the section 62, subtracting the subsequent frame 106 from the reference frame 100 results in no color or little color for most of the pixels of the difference frame 108. However, the color of the area in and around the defect 164, where the temperature difference is the greatest, are visible or more pronounced. In FIG. 17, the areas of little or no color are shown as being white whereas the areas of more pronounced color (e.g., defect 164) are shown as being darker in color. The colors may be reversed so that the areas of little or no color are dark (e.g., black, gray) while the areas of more pronounced color are lighter in color (e.g., white). The colors of FIGS. 15-17 were selected for clarity of presentation. The defect 164 may be visible in the presentation of the subsequent image 162 and may be even more visible in the presentation of the difference image 166.

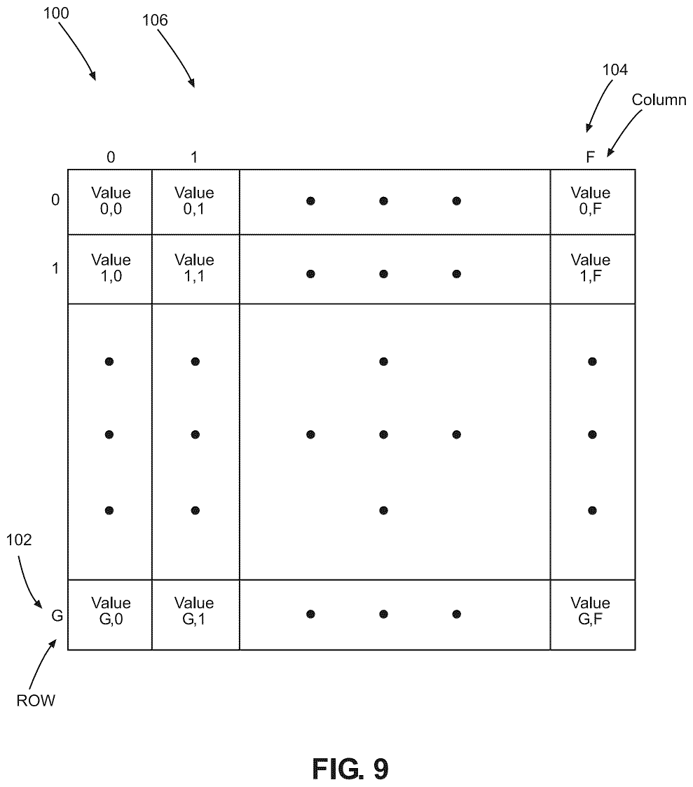

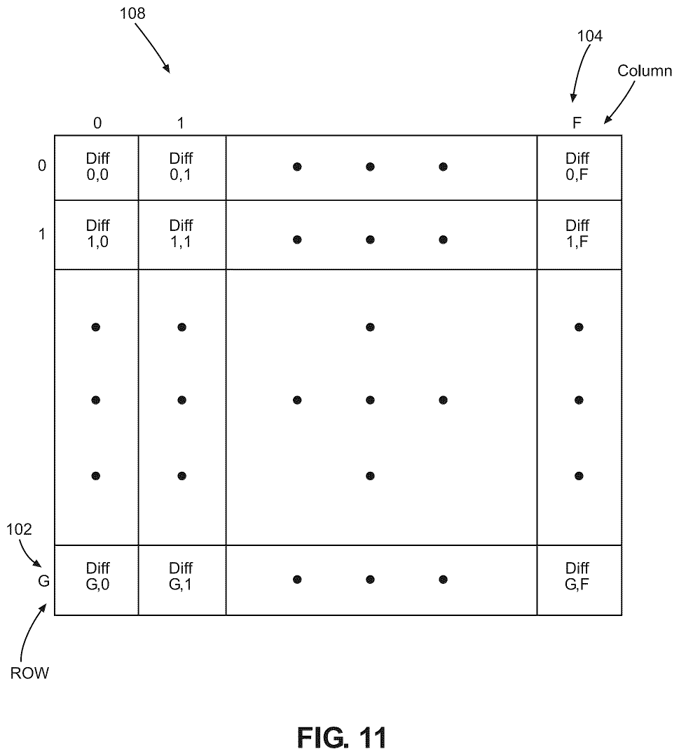

The processing circuit 42 of the computing device 40 or the server 70 may be adapted to control the display 44. The processing circuit 42 or the server 70 may provide the data to the display 44 for presentation. The display 44 may present additional information in addition to the reference image 160, the subsequent image 162 and/or the difference image 166. For example, the display 44 may present the defect data 48 and/or the captured data 49 for viewing by a user. The display 44 may further present information as to the status of an ongoing inspection, such as the total number of sections, the number of sections inspected, the number sections to be inspected, the number of defects found or other such information. The display 44 may further display the values of the pixels of a frame (e.g., value (0,0), value (0,1), value (0,2), and so forth, diff (0,0), diff (0,1), diff (0,2), and so forth), as best shown in FIGS. 9-11, as opposed to the color represented by the value of the pixel.

### R. First Example Embodiment

An embodiment of the tire inspection system may include some, but not all, of the components discussed above. Because some, but not all, of the component may be used in the system, there are many embodiments of the system. Some, but not all, of the possible embodiments are described herein.

In a first example embodiment, as best seen in FIG. 3, the system for inspecting the tire 10 includes the support 80, the tire positioner 60, the pneumatic source 30, the first infrared camera 20 for capturing frames of the tread 13 (e.g., in field-of-view 22) of the tire 10, the second infrared camera 20 for capturing frames of the sidewall 15 (e.g., in field-of-view 24) of the tire 10, the thermal modulator 50, the temperature sensor 52 and the computing device 40.

The support 80 includes the tire spindle 82, the component spindle 84 and the base 86. The tire spindle 82 is adapted to be positioned through the center of the rim 16 of the tire 10. The tire spindle 82 supports the weight of the tire 10 so that the tire 10 may be rotate around the central axis 14 of the tire spindle 82. The component spindle 84 is adapted to support the first infrared camera 20, the second infrared camera 20, the thermal modulator 50, and the temperature sensor 52. The first infrared camera 20, the second infrared camera 20, the thermal modulator 50, and the temperature sensor 52 are coupled to the component spindle so that they are oriented toward the tire 10.

The tire positioner 60 is adapted to position the tread 13 of the section 62 in the field-of-view 22 of the first infrared camera 20 and the sidewall of the section 62 in the field-of-view 24 of the second infrared camera 20. The tire positioner 60 is adapted to position the tire 10 by rotating the tire 10. The tire positioner 60 includes the manual control 66 and the rotary encoder 68. The tire positioner 60 may automatically position the tire 10 without user intervention or a user may operate manual control 66 to position the tire 10.

The rotary encoder 68 is adapted to identify the sections 62 of the tire 10. One section 62 of the tire 10 may be designated as the origin section as discussed above. Information regarding the origin section and the information from the rotary encoder 68 are used to accurately identify and position each section 62 of the tire 10. The information regarding the origin section of the tire 10 and the sections of the tire 10 as identified by the rotary encoder 68 may be stored in the captured data 49.

The pneumatic source 30 includes pneumatic collar 36, hose 32 and hose 38. The pneumatic source 30 is adapted to provide air to the tire 10 to inflate the tire 10 via the hose 32, the pneumatic collar 36 and the hose 38. The inner portion of the pneumatic collar 36 couples to the tire spindle 82. Hose 32 connects the pneumatic source 30 to the inner portion of the pneumatic collar 36. The hose 38 connects the outer portion of the pneumatic collar 36 to the valve stem 12 of the tire 10. The inner portion of the pneumatic collar 36 does not rotate with respect to the tire spindle 82, so the position and orientation of the hose 32 does not change as the tire 10 rotates between sections 62. The outer portion of the pneumatic collar 36 rotates with respect to the tire spindle 82, so that as the tire 10 rotates between sections 62, the outer portion of the pneumatic collar 36 and hose 38 rotate with the tire. The pneumatic collar 36 keeps the hose 32 and the hose 38 from becoming tangled as the tire 10 rotates. The pneumatic source 30 delivers air to the inner portion of the pneumatic collar 36 via hose 32. The inner portion of the pneumatic collar 36 delivers air to the outer portion of the pneumatic collar 36, which in turn delivers air to the tire 10 via the hose 38 to inflate the tire 10.

The computing device 40 includes the processing circuit 42, the memory 46, the communication circuit 45 and the display 44. The memory 46 stores the stored program 47, the defect data 48 and the captured data 49. The processing circuit 42 may execute the stored program 47 to control the components of the first embodiment to inspect the tire 10. The processing circuit 42 may store the data generated during the inspection in the memory 46 as the captured data 49. The processing circuit 42 may display the captured data 49 on display 44. The processing circuit 42 may transmit the captured data 49 to another device via the communication circuit 45.

For each section of the tire 10, the first infrared camera 20 captures a reference frame 100 and one or more subsequent frames 106 of the tread 13 of the tire 10. For each section of the tire 10, the second infrared camera 20 captures a reference frame 100 and one or more subsequent frames 106 of the sidewall 15 of the tire 10. The reference frames 100 and subsequent frames 106 captured by the first infrared camera 20 and the second infrared camera 20 are stored in the captured data 49.

The thermal modulator 50 (could be an infrared heater in this example) heats the section 62 of the tire 10 that is within the field-of-view 22 and/or the field-of-view 24. The thermal modulator 50 heats the section 62 while the tire 10 is been inflated by the pneumatic source 30. After the tire 10 has been inflated, the thermal modulator 50 ceases to heat the section 62.

The temperature sensor 52 senses the temperature of the section 62 of the tire 10 that is within the field-of-view 22 and/or the field-of-view 24. The temperature sensor 52 measures the temperature to which the tire 10 is been heated by the thermal modulator 50. The temperature measured by the temperature sensor 52 may be reported to the processing circuit 42. The temperature measured by the temperature sensor 52 may be stored in the captured data 49.

Once the first infrared camera 20 and/or the second infrared camera 20 have captured the reference frame 100 and one or more subsequent frames 106 for one or more of the sections 62 of the tire 10, the processing circuit 42 may compare the reference frame 100 and the one or more subsequent frames 106 for one section 62 to each other to determine whether a portion of the section 62 has decreased in temperature. Any of the comparison techniques discussed above may be used to compare the reference frame 100 to the one or more subsequent frames 106. The data generated and the data regarding any defects identified during the comparison may be stored in the defect data 48.

### S. Second Example Embodiment

In a second example embodiment, as best seen in FIG. 4, the system for inspecting the tire 10 includes the smart phone 41, which is an embodiment of the computing device 40, and pneumatic source 30 with a manual control 34. The second example embodiment permits a user to manually position the tire 10, to manually control the pneumatic source 30 to inflate the tire 10 and to position the infrared camera 20, which is integrated into the smart phone 41, to capture frames of data to detect a defect.

To perform an inspection, the user holds the tire 10 and positions the infrared camera 20 to cover a section 62 of the tire 10. The section 62 of the tire may cover the tread 13 or the sidewall 15 of the tire. The user operates (e.g., presses) the manual control 34 so that the pneumatic source 30 inflates the tire 10. When the tire 10 is inflated to a predetermined pressure, the pneumatic source 30 automatically shuts off. The user positions the smart phone 41 so that the field-of-view covers the tread 13 (e.g., field-of-view 22) or the sidewall 15 (e.g., field-of-view 24) of the section 62. The user operates the infrared camera 20 of the smart phone 41 to capture the reference frame 100 of the section 62. After period of time, the infrared camera 20 captures one or more subsequent frames of the section 62. The smart phone 41, and in particular the processing circuit 42 of the smart phone 41, compares the reference frame 100 to the one or more subsequent frames 106 to detect whether a portion of the section 62 has experienced a decrease in temperature.

The user then manually rotates the tire 10 to the next section of the tire 10. The user then holds the tire 10 while the infrared camera 20 of the smart phone 41 captures a reference frame 100 and one or more subsequent frames 106 of the next section. The user continues to manually rotate the tire 10 to the next section of the tire 10 until all sections of the tire have been inspected. As with any other implementation, the smart phone 41 may store the data captured during the inspection in the captured data 49 and any data related to defects in the defect data 48. The smart phone 41 may display any of the captured data 49 and/or the defect data 48 on the display 44 of the smart phone 41.

The second example embodiment may further include a server 70 and a database 72. The smart phone 41 may transmit the captured data 49 to the server 70 via communication link 76 and network 74. The communication link 76 may be a wireless communication link. The server 70 may compare the reference frames 100 to the one or more subsequent frames 106 to determine whether there any defects in the tire 10. If the smart phone 41 performed the defect analysis and stored the results as the defect data 48, the smart phone 41 may further transmit the defect data 48 to the server 70. The server 70 may store the captured data 49 and the defect data 48 in the database 72.

### T. Third Example Embodiment

In a third example embodiment, as best seen in FIG. 5, the system for inspecting the tire 10 includes a ramp support 88 that has a ramp-like shape. The ramp support 88 enables a vehicle 17 to pull on to the ramp support 88 so that the tire 10 may be inspected while the tire 10 and wheel 19 are mounted to the vehicle 17.

The third example embodiment of the system operates as any other embodiment of the system. The tire positioner 60 successively positions the sections 62 of the tire 10 in the field-of-view 22 or 24 of one or more infrared cameras 20. The pneumatic source 30 inflates the tire 10. The one or more infrared cameras 20 capture a reference frame 100 of the section 62 after the tire 10 is inflated. After a period of time, the one or more infrared cameras 20 capture one or more subsequent frames 106 of the section 62. The computing device 40, and in particular the processing circuit 42, compares the reference frame 100 to the one or more subsequent frames to identify a portion of the section 62 that experienced a decrease in temperature. The decrease in temperature indicates a defect 164 in the tire 10. The computing device 40 records all of the captured information in captured data 49 and all of the data regarding the defect in the defect data 48.

After the test is complete, the vehicle 17 may pull off of the ramp support 88. If no defect was found, the vehicle may continue on his journey. If a defect is found, the tire May be removed so that the defect 164 may be repaired. After the tire 10 is removed from the vehicle 17, the origin indicia 65, the section indicia 64 and the direction of rotation 67 may be used to identify the physical location of the defect 164 on the tire 10.

### U. Fourth Example Embodiment

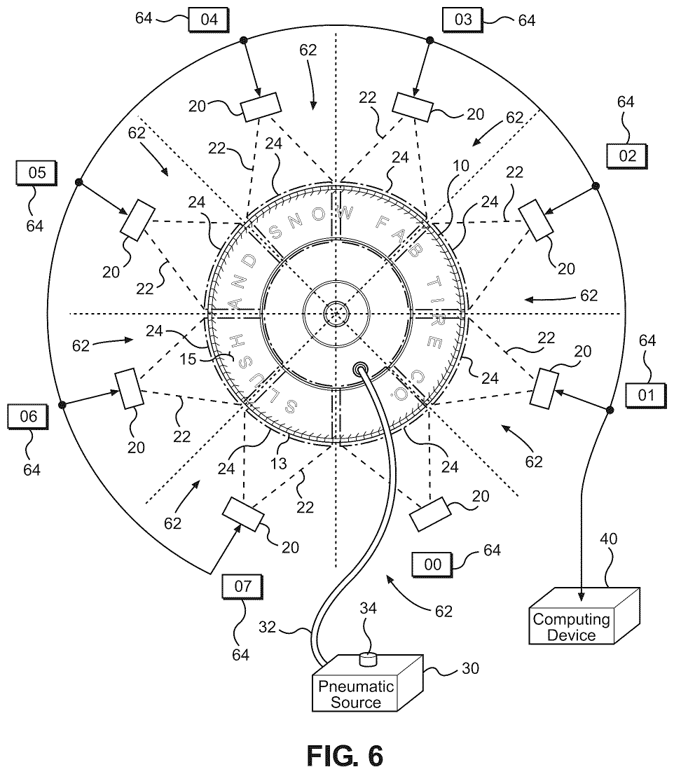

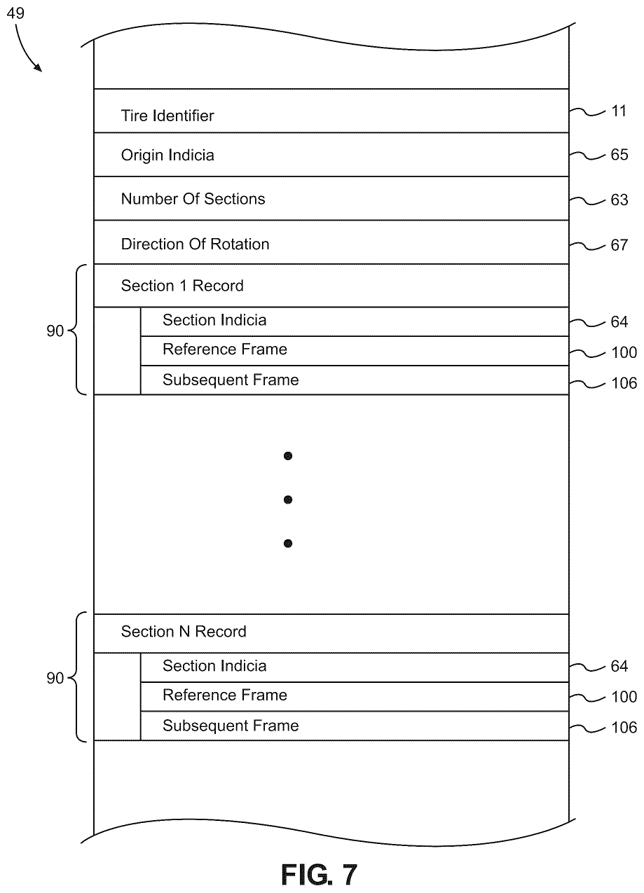

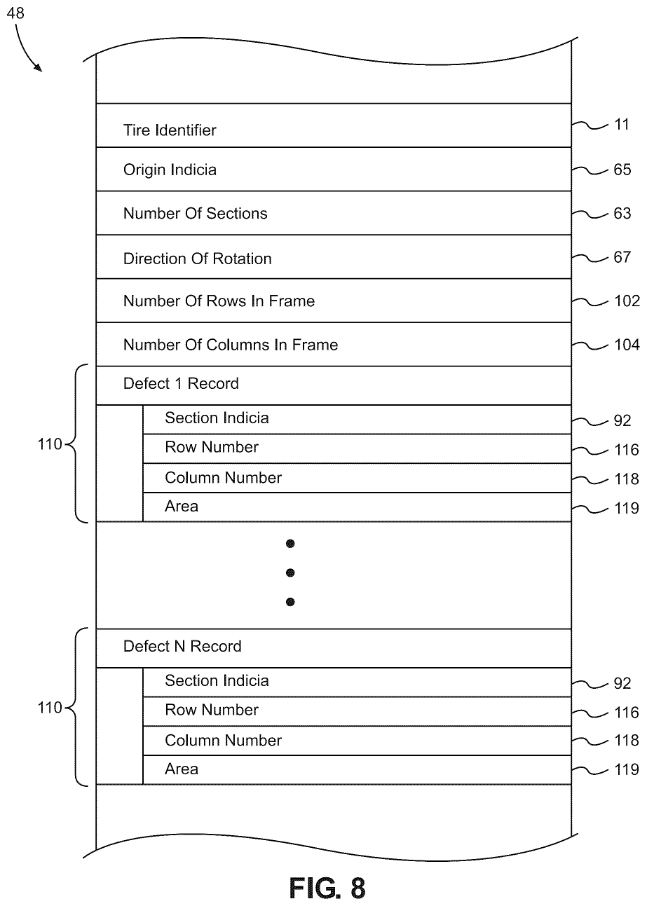

In a fourth example embodiment, as best seen in FIG. 6, the system for inspecting the tire 10 includes a plurality of infrared cameras 20, the pneumatic source 30 and the computing device 40.

The fourth example embodiment includes two infrared cameras 20 for each section of the tire 10. As shown in FIG. 2, the tire is divided into eight sections. The eight infrared cameras 20 whose field-of-view 22 is oriented to capture frame data of the tread 13 of each section 62 of the tire 10 are shown in FIG. 6. The eight infrared cameras 20 whose field-of-view 24 is oriented to capture frame data of the sidewall 15 of the tire 10 are not shown in FIG. 6 to improve the clarity of the figure.

The pneumatic source 30 inflates the tire 10. After the tire 10 100 is been inflated, each respective infrared camera 20 captures a respective reference frame of the tread 13 and the sidewall 15 of each section 62 of the tire 10. After a period of time, the infrared cameras 20 capture one or more respective subsequent frames 106 of the tread 13 and the sidewall 15 for each section 62 of the tire 10. The reference frame 100 and the one or more subsequent frames 106 from each infrared camera 20 is stored in the captured data 49. The infrared cameras 20 may capture the frames of the sections of the tire 10 at the same time, thereby making the data capture fast and efficient. A further set of eight infrared cameras 20 may be used to capture the rear sidewall 15 of the tire 10.

Once the reference frame 100 and one or more subsequent frames 106 are captured for each section 62, the data may be analyzed for each section 62 as discussed herein to identify defects 164 in the tire 10.

### V. Fifth Example Embodiment

In a fifth example embodiment, the system for inspecting the tire 10 includes the pneumatic source 30, the infrared camera 20, the tire positioner 60 and the processing circuit 42. The pneumatic source 30 is adapted to inflate the tire 10. The infrared camera 20 has a field-of-view 22. The tire positioner 60 is adapted to position the tire 10. The processing circuit 42 adapted to control the pneumatic source 30, the tire positioner 60 and the infrared camera 20.

To inspect a section 62 of the tire 10, the tire positioner 60 positions the section 62 in the field-of-view 22 of the infrared camera 20. The pneumatic source 30 inflates the tire 10. The infrared camera 20 captures a reference frame 100 of the section 62 of the tire 10 after the tire 10 is inflated. After a period of time, the infrared camera 20 captures a subsequent frame 106 of the section 62 of the tire 10. The processing circuit 42 compares the reference frame 100 to the subsequent frame 106 to detect a decrease in the temperature of the portion of the section 62. The decrease in the temperature occurs during the period of time between capture of the reference frame 100 and capture of the subsequent frame 106. The decrease in the temperature of the portion of the section 62 indicates the defect 164.

The inspection of the tire 10 may also be controlled by the processing circuit 42. The processing circuit 42 may execute a stored program 47 to perform the inspection of the tire 10. In response to executing the stored program 47, the processing circuit 42 instructs the tire positioner 60 to position a section 62 of the tire 10 in the field-of-view 22 of the infrared camera 20, instructs the pneumatic source 30 to inflate the tire 10 and instructs the infrared camera 20 to capture a reference frame 100 of the section 62. After a period of time, the processing circuit 42 instructs the infrared camera 20 to capture a subsequent frame 106 of the section 62. After the infrared camera 20 has captured the reference frame 100 and the subsequent frame 106, the processing circuit 42, still responsive to the stored program 47, compares the reference frame 100 to the subsequent frame 106 to detect a decrease in a temperature of a portion of the section 62. The decrease in the temperature occurs during the period of time between capture of the reference frame 100 and capture of the subsequent frame 106. the decrease in the temperature of the portion of the section 62 indicates the defect 164.

The first example embodiment may inspect a single section 62 of the tire 10, some of the sections of the tire 10 or all of the sections 62 of the tire 10. When inspecting some or all of the sections 62 of the tire 10, the first example embodiment of the system successively inspects the sections 62 by capturing a reference frame 100 and at least one subsequent frame 106 for each section 62 and by comparing the reference frame 100 and the at least one subsequent frame 106 to each other as discussed herein.

### W. Sixth Example Embodiment

In a sixth example embodiment, the system for inspecting the tire 10 includes the pneumatic source 30, the infrared camera 20, the tire positioner 60, the support 80, the communication circuit 45, the database 72, the server 70, the display 44, and the processing circuit 42. The pneumatic source 30 is adapted to inflate the tire 10. The infrared camera 20 has a field-of-view 22. The tire positioner 60 is adapted to position the tire 10. The support 80 is adapted to support the tire 10 for positioning by the tire positioner 60. The communication circuit 45 is adapted to transmit and receive data. The database 72 is adapted to store data. The server 70 is adapted to manage the database 72. The display 44 is adapted 16 to present images for viewing by the user. The processing circuit 42 is adapted to control the pneumatic source 30, the tire positioner 60, the infrared camera 20 and the communication circuit 45.

To inspect a section 62 of the tire 10, the tire positioner 60 positions the section 62 of the tire 10 in the field-of-view 22 of the infrared camera 20. The pneumatic source 30 inflates the tire 10. The infrared camera 20 captures a reference frame 100 of the section 62 of the tire 10 after the tire 10 is inflated. After a period of time, the infrared camera 20 captures a subsequent frame 106 of the section 62 of the tire 10. The processing circuit 42 transmits the reference frame 100 and the subsequent frame 106 to the server 70 via the communication circuit 45. The server 70 compares the reference frame 100 to the subsequent frame 106 to detect a decrease in the temperature of a portion of the section 62. The decrease in the temperature occurs during the period of time between capture of the reference frame 100 and capture of the subsequent frame 106. The decrease in the temperature of the portion of the section 62 indicates a defect 164. The server 70 stores at least one of the reference frame 100, the subsequent frame 106 and the result of comparing in the database 72. The display 44 presents an image of the defect 164 to the user.

### X. Seventh Example Embodiment

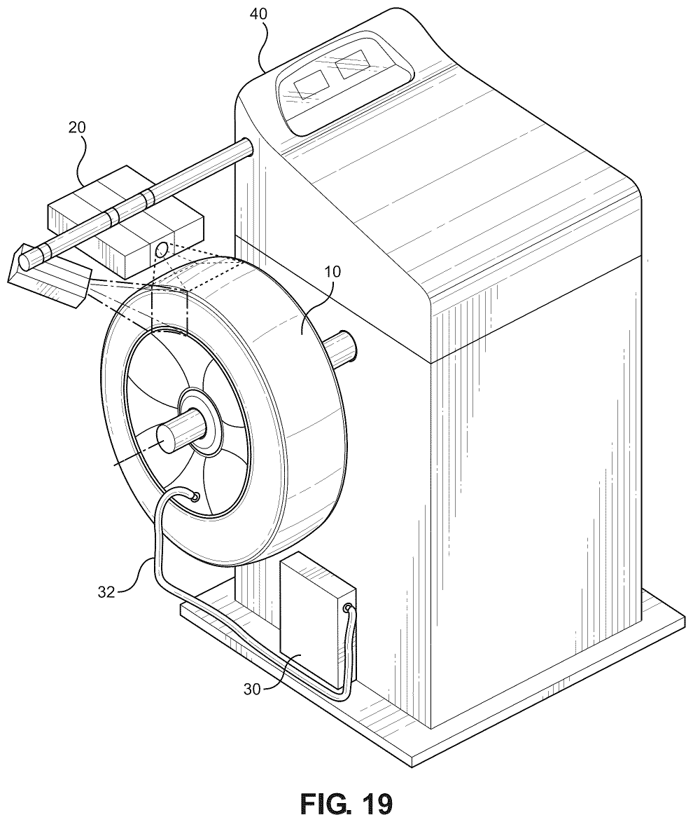

In a seventh example embodiment, as best seen in FIG. 19 (in view of FIG. 1), the system for inspecting the tire 10 includes a wheel balancer 200, a pneumatic source 30, an 8 infrared camera 20, a computing device 40, and a thermal modulator 50. This embodiment enables the detection of tire defects while the tire 10 remains mounted on the wheel balancer 200, allowing for simultaneous balancing and defect detection. The wheel balancer 200 supports and rotates the tire 10 during the inspection process, utilizing a motorized spindle 202 that can rotate the tire 10 at controlled speeds.

The pneumatic source 30 is adapted to inflate the tire 10 to a predetermined pressure. It includes a hose 32 that connects to the valve stem of the tire 10. The pneumatic source 30 can be controlled manually or automatically by the computing device 40 to ensure the tire 10 reaches the required pressure for accurate defect detection. The pneumatic source 30 can also be controlled by the computing device 40 along with the thermal modulator 50 to regulate the temperature of the air used for inflation, ensuring the tire is either heated or cooled as needed for the inspection process.

As noted above, the computing device 40 can control the pneumatic source 30 and the thermal modulator 50 to perform various methods of defect detection. In one embodiment, the tire is heated externally by the thermal modulator 50. Any defects produce local temperature differences which can be detected by the infrared camera 20. In another embodiment, the air used to inflate the tire 10 is heated or cooled, but not the tire itself. Defects are exposed when the thermally regulated air at the defect is detected by the infrared camera 20. In yet another example, both the tire and the air are subject to thermal manipulation. For example, the tire can be heated by the thermal modulator 50, while the air used to inflate the tire is cooled by another thermal modulator 50. Thus, the system can include multiple thermal modulators in some instances.

In one example, the infrared camera 20 is positioned to capture thermal images of the tire 10 as it rotates on the wheel balancer 200. The infrared camera 20 has a field-of-view that can cover both the tread and the sidewall sections of the tire 10. The captured images are sent to the computing device 40, which utilizes blob detection algorithms to analyze the thermal images. By comparing the reference frame captured immediately after inflation to subsequent frames taken after a period, the system can detect localized temperature changes indicating potential defects.

The computing device 40, equipped with a processing circuit 42 and memory 46, controls the entire inspection process. It automates the inflation, rotation, and thermal modulation of the tire 10, as well as the capture and analysis of thermal images. The device stores all relevant data, including reference and subsequent frames, defect data, and inspection results, which can be displayed for the user and archived for future reference. This integrated system on the wheel balancer 200 streamlines the tire inspection process, providing efficient and accurate detection of defects while balancing the tire 10.

### Y. Eighth Example Embodiment

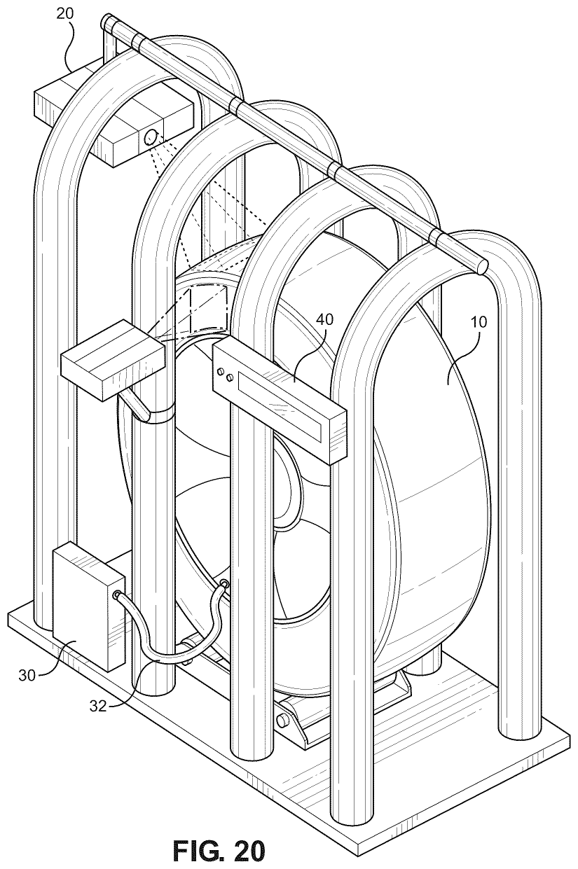

In an eighth example embodiment, as best seen in FIG. 20 (in view of FIG. 1), the system for inspecting the tire 10 includes a tire cage 300, a pneumatic source 30, an infrared camera 20, a computing device 40, and a thermal modulator 50. This embodiment enables the detection of tire defects while the tire 10 is securely mounted within the tire cage 300, providing a safe and controlled environment for the inspection process.

The tire cage 300 is designed to securely hold the tire 10 in place during the inspection. It includes adjustable clamps 302 that can accommodate various tire sizes and ensure that the tire remains stationary during inflation and thermal imaging. The tire cage 300 enhances safety by containing the tire 10 and any potential debris in the event of a blowout or other malfunction during the inspection process.

The pneumatic source 30 is used to inflate the tire 10 to a predetermined pressure. It includes a hose 32 that connects to the valve stem 12 of the tire 10. The pneumatic source 30 can be controlled either manually or automatically by the computing device 40, ensuring that the tire 10 is inflated to the correct pressure for accurate defect detection. Additionally, the pneumatic source 30 can work in conjunction with the thermal modulator 50 to heat or cool the air used for inflation, thereby aiding in the detection of defects.

The infrared camera 20 is positioned within the tire cage 300 to capture thermal images of the tire 10. The camera's field-of-view covers critical areas of the tire, including the tread and sidewalls. As the tire 10 is inflated and subsequently observed, the infrared camera 20 captures a series of thermal images. These images are analyzed by the computing device 40 using blob detection algorithms to identify regions where temperature changes indicate the presence of defects.

The thermal modulator 50 is responsible for adjusting the temperature of the tire 10 to enhance defect detection. It may utilize infrared heaters, hot air blowers, or refrigerant-based cooling systems to create a temperature differential. This temperature differential accentuates areas where air escapes through defects, making them more visible in the thermal images captured by the infrared camera 20.

The computing device 40, equipped with a processing circuit 42 and memory 46, orchestrates the entire inspection process within the tire cage 300. It automates the inflation, thermal modulation, and image capture processes. The computing device 40 stores all relevant data, including reference frames, subsequent frames, defect data, and inspection results. This data can be displayed for the user and archived for future reference, providing a comprehensive record of the tire's condition. This integrated system within the tire cage 300 ensures a thorough and safe inspection process, effectively identifying and documenting tire defects.

### Z. Ninth Example Embodiment

In another example, a system for inspecting a tire is integrated with a tire changing machine. This embodiment combines the functionalities of a tire changer and a tire defect detection system, allowing for seamless tire inspection during the tire changing process.

A tire changing machine is a piece of equipment in automotive repair shops designed to efficiently remove and install tires on vehicle rims. This machine automates the labor-intensive process of changing tires, significantly reducing the time and effort required by technicians. It typically features a motorized mechanism that can securely grip the rim, demount the old tire, and mount the new one with precision. The machine often includes various tools, such as bead breakers to separate the tire from the rim and arms that assist in positioning and aligning the tire during installation.

The tire changing machine is equipped with various components that facilitate both the removal and installation of tires on wheels, as well as the detection of defects in the tires. The system includes a pneumatic source, an infrared camera, a computing device, a thermal modulator, and a tire positioner. The pneumatic source is adapted to inflate the tire with an expanding gas that is either heated or cooled by the thermal modulator. The pneumatic source ensures the tire is inflated to a predetermined pressure, necessary for accurate defect detection.

The thermal modulator can either heat or cool the tire or the air pumped into the tire. This helps create a temperature differential that enhances the detection of defects by the infrared camera. The thermal modulator can include components such as infrared heaters, hot air blowers, or cooling systems. The infrared camera captures thermal images of the tire to detect localized cooling or heating that indicates defects. The camera is positioned to monitor the tire 10 while it is mounted on the tire changing machine.

The computing device processes the thermal images captured by the infrared camera. It uses blob detection algorithms to analyze the images and identify areas where temperature changes indicate potential defects. The computing device also controls the overall operation of the inspection system, including the pneumatic source, the thermal modulator, and the tire positioner. The tire positioner ensures that the tire is correctly aligned and positioned during the inspection process. It can rotate the tire to different sections for a comprehensive inspection, ensuring that the entire surface of the tire is monitored for defects.

### Aa. Tenth Example Embodiment

In another example, a system for inspecting a tire is integrated with a tire spreader. This embodiment combines the functionalities of a tire spreader and a tire defect detection system, enabling thorough inspection of tires during the spreading process.

For context, a tire spreader is a tool used in automotive repair and maintenance shops for inspecting, repairing, and servicing tires. It holds the tire in a spread position, allowing technicians to thoroughly inspect the inner and outer surfaces for defects, damage, or wear. This is especially useful for identifying issues such as punctures, cuts, or separations that may not be visible when the tire is in its normal shape.

By spreading the tire, the tool provides better access to the inside of the tire, facilitating repairs. Technicians can easily reach and patch punctures, apply inner liners, or perform other necessary repairs to ensure the tire's integrity and safety. Additionally, the spreader enables efficient cleaning and preparation of the tire before repair, ensuring that the adhesive patches or liners bond properly. This tool is essential for maintaining tire safety and performance, making it a staple in any tire service operation.

The tire spreader is equipped with various components that facilitate both the spreading of tires to expose the inner surfaces and the detection of defects in the tires. The system includes a pneumatic source, an infrared camera, a computing device, a thermal modulator, and a tire positioner. The pneumatic source is adapted to inflate the tire with an expanding gas that is either heated or cooled by the thermal modulator. The pneumatic source ensures the tire is inflated to a predetermined pressure, necessary for accurate defect detection.

The thermal modulator can either heat or cool the tire or the air pumped into the tire. This helps create a temperature differential that enhances the detection of defects by the infrared camera. The thermal modulator can include components such as infrared heaters, hot air blowers, or cooling systems. The infrared camera captures thermal images of the tire 10 to detect localized cooling or heating that indicates defects. The camera is positioned to monitor the tire while it is mounted on the tire spreader.

The computing device processes the thermal images captured by the infrared camera. It uses blob detection algorithms to analyze the images and identify areas where temperature changes indicate potential defects. The computing device also controls the overall operation of the inspection system, including the pneumatic source, the thermal modulator, and the tire positioner.

The tire positioner ensures that the tire is correctly aligned and positioned during the inspection process. It can rotate the tire to different sections for a comprehensive inspection, ensuring that the entire surface of the tire is monitored for defects. The tire spreader is designed to hold the tire in a spread position, allowing for a detailed inspection of both the inner and outer surfaces. This configuration ensures that any defects, even those not visible from the outside, can be detected effectively.

### Bb. Example Method for Frame Capture

All embodiments of the system include capturing reference frames 100 and one or more subsequent frames 106. Some embodiments analyze only the one or more subsequent frames 106 to identify the defects 164. Other embodiments compare the one or more subsequent frames 106 to the reference frame 100 to identify the defects 164. Other embodiments subtract one of the subsequent frames 106 from the reference frame 100 to form the difference frame 108 and the difference frame 108 is analyzed to identify the defects 164.

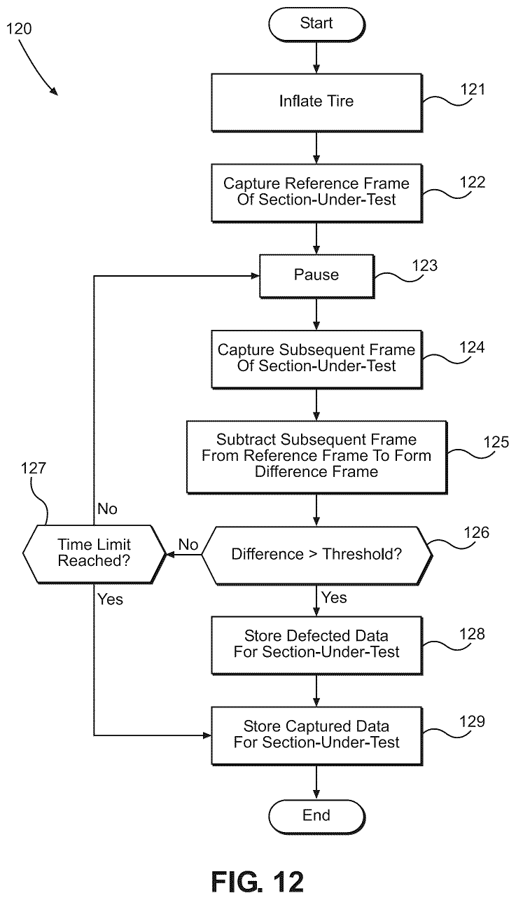

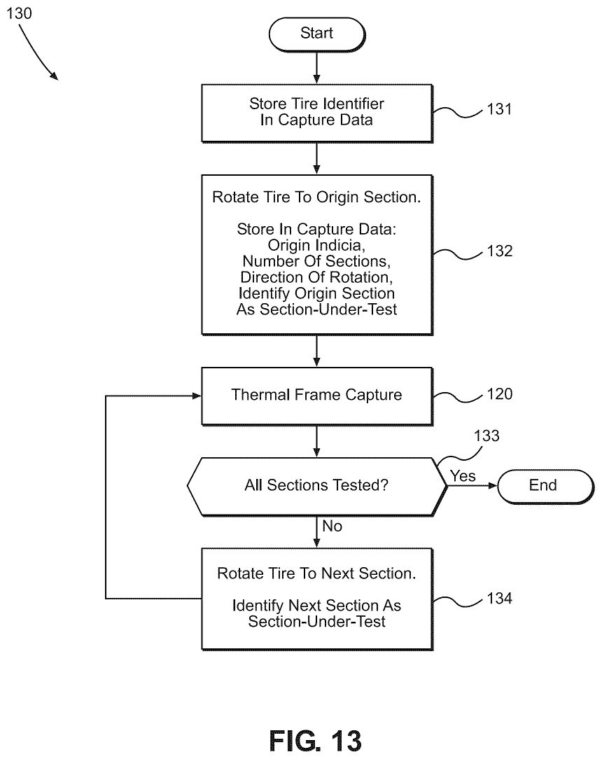

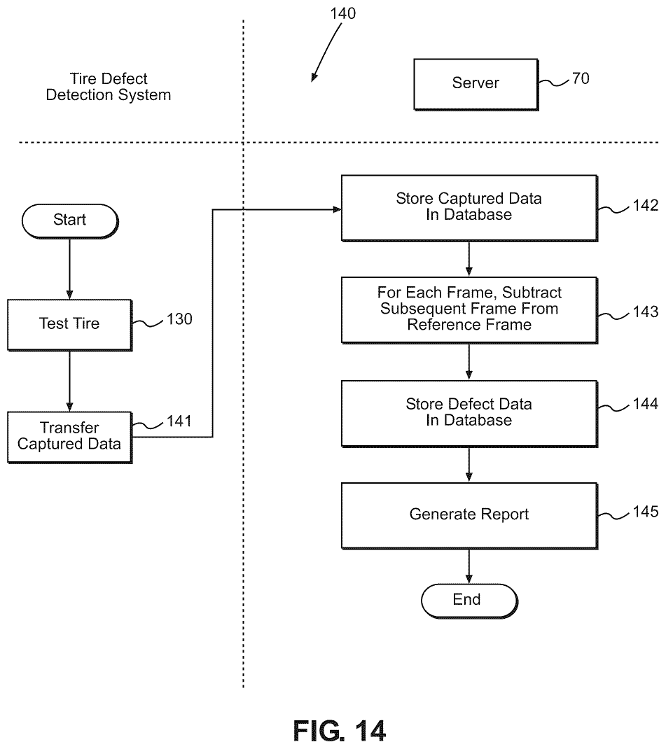

The example method of FIG. 12 is an example of a method for capturing a reference frame 100, capturing a subsequent frame 106, subtracting the subsequent frame 106 from the reference frame 100 to form the difference frame 108 and storing the reference frame 100, the subsequent frame 106, and the difference frame 108 for analysis. The method frame capture 120 includes inflate 121, capture reference 122, pause 123, capture subsequent 124, subtract 125, threshold 139, time limit 127, store 128 and identify 129.

In the step inflate 121, the pneumatic source 30 inflates the tire 10. The pneumatic source 30 inflates the tire 10 to a pre-determined pressure. The pneumatic source 30 stops inflating the tire 10 when the pressure of the tire 10 reaches the predetermined pressure.

In the step capture reference 122, the infrared camera 20 captures a reference frame 100 of the section 62 of the tire 10 covered by the field-of-view (e.g., 22, 24) of the infrared camera 20. The section 62 which is presently in the field-of-view of the infrared camera 20 is referred to as the section-under-test.

In the step pause 123, the performance of the method pauses for a period of time. The period of time is to allow air to escape through the tire 10 if there is a defect in the tire 10. Air that escapes to the defect will cool the area of the tire 10 around the defect 164 thereby enabling detection of the defect 164.

In the step capture subsequent 124, performed after the step pause 123, the infrared camera 20 captures a subsequent frame 106 of the section-under-test.

In the step subtract 125, the subsequent frame 106 is subtracted from the reference frame 100 to find a different between the reference frame 100 and the subsequent frame 106. Because the infrared camera 20 captures thermal data of the section-under-test, a difference between the reference frame 100 and the subsequent frame 106 will include the difference in temperature. In particular, the difference in temperature may occur in the portion of the section-under-test where there is a defect 164.

In the step threshold 139, the difference between the reference frame 100 and the subsequent frame 106 is compared to a threshold. Comparing the difference to a threshold may be used to determine whether the difference indicates a defect 164. If there is a defect 164 located in the section-under-test, the air escaping through the defect 164 will decrease the portion of the tire 10 around the defect 164 as compared to the remainder of the section-under-test. The defect 164 may result in a decrease in the temperature of the portion of the section-under-test as small as 0.1 degrees. Depending on the size of the defect 164, the temperature may decrease several (e.g., more than two) degrees. The temperature may be measured as Celsius or Fahrenheit degrees. If the decrease in temperature is less than the threshold, then it is likely that there is no defect 164 or that there has not been enough time for the air escaping from the defect 164 to sufficiently cool the tire.

If the difference is greater than the threshold, then it is likely that a defect 164 exists, so execution of the method continues with the step store 128. If the difference is less than the threshold, then it is likely that a defect 164 does not exist or there has not been a sufficient lapse of time to cool the tire 10 around the defect 164. If the difference is less than the threshold, execution of the method continues with the step time limit 127.

In the step time limit 127, the method determines whether a time limit for determining whether a defect 164 exists in the section-under-test has been reached. The time limit is the maximum amount of time allowed to find a difference in temperature that is greater than the threshold. The time limit is a maximum amount of time that is allowed for air to escape from the defect 164 in the tire 10 to lower the temperature of the portion of the section-under-test. If the defect 164 is not in the section-under-test, the tire 10 may completely deflate without changing the temperature of a portion of the section-under-test. If the time limit has not been reached, then execution of the method continues with the step pause 123. If the time limit has been reached, then execution of the method continues with the step identify 129.

In the step store 128, the defect data for the section-under-test is stored in the defect data 48. If the defect data is anything more than the result of subtracting the subsequent frame 106 from the reference frame 100 as performed in the step subtract 125, then the step store 128 performs the additional analysis required to determine the defect data 48 for the section-under-test. For example, the step store 128 may include performing blob detection on the reference frame 100, the subsequent frame 106 and/or the difference frame 108. The defect data 48 may include the location (e.g., row, column) of the defect 164 in the section-under-test and thermal images of the defect 164.

In the step identify 129, the results of subtracting the subsequent frame 106 from the reference frame 100 as performed in the step subtract 125, is identified as the difference frame 108. In the step identify 129, the data captured while performing the method, including the reference frame 100, the subsequent frame 106 and the difference frame 108 are stored in the captured data 49.

### Cc. Example Methods of Tire Inspection

The tire 10 may be inspected using various embodiments of inspection methods. In a first example embodiment, the tire test method 130 includes store 131, rotate to origin 132, frame capture 120, all sections 133, and rotate 134.

In the step store 131, the tire identifier 11 for the tire-under-test is stored in the captured data 49. The tire identifier 11 may include the serial number of the tire, as discussed above. The tire identifier 11 may include photographs of the tire sufficient to identify the tire.

In the step rotate to origin 132, the tire 10 is rotated to position the origin section of the tire 10 in the field of view (e.g., 22, 24) of the infrared camera 20. Information regarding the origin section including the origin indicia 65, the number of sections 63 and the direction of rotation 67 are stored in the captured data 49. Once positioned in the field of view of infrared camera 20, the origin section is identified as the section-under-test.

In the frame capture 120, all of the steps of the method frame capture 120 are performed to capture the reference frame 100, the subsequent frame 106 and the difference frame 108 each of which is stored in the captured data 49 in a section record 90 for the section-under-test. The frame capture 120 further identifies any defects 164 in the section-under-test, if any, and stores the information regarding the defect 164 in the defect data 48.

In the step all sections 133, it is determined whether all sections 62 of the tire have had a turn at being the section-under-test. The number of sections 62 was determined in the step rotate to origin 132, so it possible to determine when all of the sections 62 of the tire 10 have had their turn being the section-under-test and the data has been collected for each section 62 of the tire 10.

If all the sections of the tire have been tested, then the method is finished and ends. If all of the sections of the tire 10 have not been tested, then execution continues with the step rotate 134.

In the step rotate 134, the tire is rotated to the next section that has not been tested and this next section becomes the section-under-test. Execution then moves to the step frame capture 120 and data is captured regarding this new section-under-test and the defects 164, if any, are identified.

A second example embodiment of a method for inspecting the tire 10 includes inflating the tire 10, capturing a reference frame 100, capturing a subsequent frame 106 and comparing the reference frame 100 to the subsequent frame 106. The step of inflating the tire 10 includes inflating the tire 10 to a pre-determined pressure.

The step of capturing the reference frame 100 includes capturing the reference frame 100 of a section 62 of the tire using an infrared camera 20. The section 62 of the tire 10 is the section 62 that is presently within the field-of-view (e.g., 22, 24) of the infrared camera 20. The infrared camera 20 captures the reference frame 100 shortly or immediately after the tire 10 is inflated. The step of capturing the subsequent frame 106 includes after a period of time includes capturing the subsequent frame 106 of the section 62 using the infrared camera 20.

The step comparing the reference frame 100 to the subsequent frame 106 includes comparing the reference frame 100 to the subsequent frame 106 to detect a decrease in a temperature of a portion of the section 62. The decrease in the temperature occurs during the period of time between capture of the reference frame 100 and capture of the subsequent frame 106. The decrease in the temperature of the portion of the section 62 indicates a defect 164.

### DD. Example Methods of Defect Detection by a Computing Device

As discussed above, a system for inspecting the tire 10 may capture data regarding the sections 62 of the tire 10, then provide the captured data 49 to the server 70 (or other example computing system such as an end user computer or terminal) to analyze the data to determine whether a defect 164 exists in any of the sections 62 of the tire.

In an example method, the system for inspecting the tire 10 performs the example method test tire 130, which captures data regarding each section of the tire 10. The example method frame capture 120, discussed above, is executed by the example method test tire 130. The example method frame capture 120 includes detecting whether the section-under-test includes a defect 164. Since in this example method, the server 70 will use the captured data 49 to determine whether there any defects 164 in the tire 10, any defect detection done by the example method frame capture 120 may be omitted. However, the example method frame capture 120 may include determining the difference frame 108 (e.g., subtract 125), storing the difference frame 108 in the captured data 49 (e.g., identify 129) and sending the differential frame to the server 70 as part of the captured data 49.

In the step transfer 141, the system for inspecting the tire 10 transfers the data captured while inspecting the tire 10 to the server 70. The data transferred may include only the captured data 49 if defect detection is omitted from the example method test tire 130. The data transferred may include the captured data 49 and the defect data 48 if defect detection is included in the example method test tire 130. The data may be transferred from the system for inspecting the tire 10 using the communication circuit 45, the communication link 76 and the network 74.

In the step store 142, the server 70 receives the data from the system and stores the data in the database 72. If the data received from the system includes only the captured data 49, the server 70 stores only the captured data 49 in the database 72. If the data received from the system includes the captured data 49 and the defect data 48, the server 70 stores the captured data 49 and the defect data 48 in the database 72.

If the data received from the system does not include the difference frame 108 or the defect data 48, the step subtract 125 is performed by the server 70 to determine the difference frame 108 and the defect data 48. In the event that the server 70 performs the step subtract 125, the server 70 accesses the captured data 49 from the database 72 to perform the step subtract 125.

If the server 70 performs the step subtract 125, then the server 70 executes the step store 128 to store the defect data 48 in the database 72. If the server 70 received the defect data 48 from the system, then the defect data 48 is already stored in the database 72.

In the step generate 145, the server 70 generates reports regarding the tire 10, regarding the captured data 49 and/or the defect data 48. The report may include any or all data from the captured data 49 and/or the defect data 48. The report may include images of the difference frame 108 and/or any other frame created during defect analysis. The frames that are part of the report may be presented on the display 44 for viewing by a user.

The report may further use historical data stored for a specific tire 10 to generate the report. The step generate 145 may use any analysis techniques and/or mathematical techniques (e.g., statistics) to generate the report to provide information regarding the tire 10 to the user.

### EE. Example Method for Frame Subtraction

As discussed in several places herein, the subsequent frame 106 may be subtracted from the reference frame 100. For example, the step 125 subtracts the subsequent frame 106 from the reference frame 100.

The example method subtraction 150 is an example method for performing pixel-wise subtraction the subsequent frame 106 from the reference frame 100. The method subtraction 150 is described as pseudocode for execution by the processing circuit 42. The pseudocode includes the for statement 151 that steps through each row of the frame data (e.g., reference frame 100, subsequent frame 106) and a for statement 152 that steps through each column of the frame data. The mathematical formula described as Diff (Row, Column) in step 153 performs the actual subtraction of the value of a pixel (e.g., value (Row, Column) from the subsequent frame 106 from the value (e.g., value (Row, Column) of the corresponding pixel from the reference frame 100. The result other subtraction may be stored in the corresponding pixel of the difference frame 108.

The for step 151 addresses each row of the reference frame 100 and subsequent frame 106 in order from zero to the last row, which is identified as row G. The for step 152 addresses each column of the reference frame 100 and subsequent frame 106 in order from zero to the last column, which is identified as column F. The for steps 151 and 152 access one pixel (e.g., pixel Row, Column) of the reference frame 100 and the corresponding pixel (e.g., pixel Row, Column) of the subsequent frame 106 at a time. For example, when the method subtraction 150 begins, Row is equal to zero and Column=zero, so the value of the (0, 0) pixel in the subsequent frame 106 is subtracted from the value of the (0, 0) pixel in the reference frame 100. The result of the subtraction is stored in the (0, 0) pixel of the difference frame 108.

The value of Column variable is incremented so that the next pixel value of the (0, 1) pixel in the subsequent frame 106 is subtracted from the value of the (0, 1) pixel in the reference frame 100. The result of the subtraction is stored in the (0, 1) pixel of the difference frame 108. The Column variable is incremented until it is equal to F. After subtraction of the value of the (0, F) pixel from the subsequent frame 106 is subtracted from the value of the (0, F) pixel in the reference frame 100 and the result stored in the (0, F) pixel of the difference frame 108, the Column variable is set to zero and the Row variable is incremented to one, so the value of the (1, 0) pixel in the subsequent frame 106 is subtracted from the value of the (1, 0) pixel in the reference frame 100. The result of the subtraction is stored in the (1, 0) pixel of the difference frame 108. The Column and Row variables are incremented and the difference taken until the Column and Row variables reach the (G, F) pixel, which is last pixel in the frame.

Upon completion of the method subtraction 150, the value of each pixel in the subsequent frame 106 has been subtracted from the corresponding value of the pixel in the reference frame 100 and the result has been stored in the corresponding pixel of the difference frame 108.

### FF. Operation of Preferred Embodiment

In use, referring to a fully automated system, a user places the tire 10 on the tire spindle 82 and positions the tire 10 over the tire positioner 60. The user slides the pneumatic collar 36 over the tire spindle 82 and connects the hose 38 to the valve stem 12 of the tire 10. The tire positioner 60 rotates the tire 10 to position a predefined origin section (e.g., valve stem 12 in center of the section) in the field-of-view 22 or 24. Alternatively, the system may record origin indicia thereby making any section of the tire the origin section. Once the tire has been positioned so that the origin section is positioned in field-of-view 22 or 24 of the first infrared camera 20 camera or the second infrared camera 20, the rotary encoder 68 may be reset so that the section indicia 64 of the origin section is zero (e.g., 00). The section 62 of the tire 10 that is positioned in the field-of-view 22 or 24 is referred to as the section-under-test.

Pneumatic source 30 inflates the tire 10. As the tire 10 is being inflated, the thermal modulator 50 heats or cools the section-under-test. Shortly or immediately after the tire 10 is inflated, the temperature sensor 52 detects the temperature of the section-under-test. Further, the first infrared camera 20 and/or the second infrared camera 20 capture a reference frame 100 of the tread 13 and/or sidewall 15 respectively of the section-under-test. The system waits for a period of time to allow air to escape from any defects 164. After the period of time, first infrared camera 20 and/or the second infrared camera 20 capture a subsequent frame 106 of the tread 13 and/or the sidewall 15 of the section-under-test.

After the subsequent frame 106 has been captured, the tire positioner 60 rotates the tire 10 to a next section of the tire 10, so that the next section becomes the section-under-test. The tire positioner 60 knows the number of sections for the tire 10 or the size of the sections of tire 10, so the tire positioner 60 may accurately rotate the tire to the next section. The system then repeats inflating the tire 10 and capturing the reference frame 100 and the subsequent frame 106.

The system rotates the tire 10 and captures the frame data (e.g., reference frame 100, subsequent frame 106) until all sections of the tire 10 have been inspected. The processing circuit 42 of the computing device 40 may store the captured data as the captured data 49 in the memory 46. The processing circuit 42 may also compare the subsequent frame 106 to the reference frame for each section 62 to determine whether a defect 164 exists in any of the sections 62. The processing circuit 42 may store data regarding any defects 164 detected in the defect data 48 in the memory 46.

The system may present any data on the display 44 (not shown) for viewing by the user. The data presented may include images, such as the reference image 160, the subsequent image 162, the difference image 166 and/or any data determined by the processing circuit 42 including any or all defect data 48.

A system may also be used for identifying the locations of any defects 164 after the initial inspection of the tire 10. In use, the user may place the tire 10 and the pneumatic collar 36 on the tire spindle 82. The processing circuit 42 may access the captured data 49 to retrieve the origin indicia 65, the number of sections 63 and the direction of rotation 67. The system rotates the tire 10 until the origin section is positioned in the field-of-view 22 of the first infrared camera 20 or the field-of-view 24 of the second infrared camera 20. The system may include a vision system for identifying features of the tire 10 so that the tire may be rotated to position the origin section in the fields-of-view of the first infrared camera 20 and/or the second infrared camera 20. The processing circuit 42 accesses the defect data 48 and uses the defect records 110 to rotate the tire to each section that has a defect 164. The display may present an image of the section of the tire 10 so that the defect 164 and the location of the defect in the section 62 may be seen by the user.

Unless otherwise defined, all technical and scientific terms used herein have the same meaning as commonly understood by one of ordinary skill in the art to which this invention belongs. Although methods and materials similar to or equivalent to those described herein can be used in the practice or testing of the various embodiments of the present disclosure, suitable methods and materials are described above. All patent applications, patents, and printed publications cited herein are incorporated herein by reference in their entireties, except for any definitions, subject matter disclaimers or disavowals, and except to the extent that the incorporated material is inconsistent with the express disclosure herein, in which case the language in this disclosure controls. The various embodiments of the present disclosure may be embodied in other specific forms without departing from the spirit or essential attributes thereof, and it is therefore desired that the various embodiments in the present disclosure be considered in all respects as illustrative and not restrictive. Any headings utilized within the description are for convenience only and have no legal or limiting effect.

What is claimed is:

1. A method for inspecting a tire for a defect, the method comprising:
inflating the tire with a gas;
heating or cooling the tire or the gas with and a thermal modulator;
pausing for a predetermined period;
capturing a thermal image of a section of the tire after the pause;
comparing the thermal image of the section using a blob detection method to identify temperature differences within the section;
determining that a defect exists and storing defect data if the temperature difference exceeds a threshold;
determining whether an elapsed time from a start of the pause exceeds a threshold amount of time if the temperature difference is below the threshold; and
repeating the capturing, comparing, and determining steps if the elapsed time does not exceed the threshold amount of time.

2. The method of claim 1, further comprising defining the tire as having a plurality of equivalent sections with each equivalent section having a same number of degrees relative to a 360 degree circumference of the tire.

3. The method of claim 1, wherein inflating the tire includes inflating the tire to a predetermined pressure.

4. The method of claim 1, further comprising heating or cooling the tire while inflating the tire.

5. The method of claim 1, further comprising heating a section of the tire while inflating the tire.

6. The method of claim 1, wherein comparing the thermal image using the blob detection method includes performing analysis on the defect data of the section.

7. The method of claim 1, wherein the defect data includes the temperature difference.

8. The method of claim 7, wherein the defect data additionally includes a location of the defect in the section.

9. The method of claim 8, wherein the location of the defect is identified by an assigned column and row within the section.

10. The method of claim 1, further comprising rotating the tire to a new section and repeating the inflating, pausing, capturing, comparing, and determining steps for the new section.

11. The method of claim 10, wherein rotating the tire comprises manually rotating the tire.

12. The method of claim 10, wherein rotating the tire comprises automatically rotating the tire.

13. The method of claim 10, wherein rotating the tire occurs while the tire is removed from a vehicle.

14. The method of claim 10, wherein rotating the tire occurs while the tire remains attached to a vehicle.

15. The method of claim 1, wherein the threshold amount of time is a predetermined maximum amount of time allowed for air to escape from the defect.

16. The method of claim 1, further comprising storing non-defect data, including the thermal image, the temperature difference, and the elapsed time, if no defect is detected.

17. The method of claim 1, further comprising generating an image of the tire with any determined defects and displaying the image.

18. The method of claim 1, further comprising repeating the inflating, pausing, capturing, comparing, and determining steps for each of a plurality of sections of the tire, and automatically rotating the tire to a position where any determined defect is presented for repair.

19. A system for inspecting a tire for defects, the system comprising:
a pneumatic source configured to inflate the tire with a fluid;
an infrared camera having a field-of-view of a section of the tire;
a thermal modulator configured to heat or cool the fluid and/or the tire; and
a computing device configured to:
control the pneumatic source, the infrared camera, and the thermal modulator; and
use a blob detection method to compare thermal images to identify temperature differences indicating defects.

20. The method of claim 19, wherein the system additionally includes a tire positioner controlled by the computing device to rotate the tire to place the section in the field-of-view of the infrared camera.
